SEQUENCE LISTING (8 sequences)

SEQ ID NO: 1 - protein, 227 aa, Artificial
  IgG1 Mutation of N297 to Alanine
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 227 aa, Artificial
  IgG1 Mutation of Leucine 234 to Alanine and Leucine 235 to Alanine
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 227 aa, Artificial
  IgG1 Mutation of Proline 329 to Glycine
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 227 aa, Artificial
  IgG1 Mutation of Leucine 235 to Glutamic Acid
DKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 227 aa, Artificial
  IgG1 Mutation of Leucine 234 to Alanine, Leucine 235 to Alanine, and Proline 329 to Glycine
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 326 aa, Artificial
  IgG2a Mutation of Valine 235 to Alanine, Glycine 237 to Alanine, Proline 238 to Serine, Histidine 268 to Alanine, Valine 309 to Leucine, Alanine 330 to Serine, and Proline 331 to Serine
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 7 - protein, 226 aa, Artificial
  IgG4 Mutation of Serine 228 to Proline and Leucine 235 to Glutamic Acid
YGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 8 - protein, 226 aa, Artificial
  IgG4 Mutation of Serine 228 to Proline, Leucine 235 to Glutamic Acid, and Proline 329 to Glycine
YGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

(19) United States
(12) Patent Application Publication
(10) Pub. No.: US 2020/0095323 A1
(43) Pub. Date: Mar. 26, 2020

(54) MITIGATING Fc-Fc RECEPTOR INTERACTIONS IN CANCER IMMUNOTHERAPY

(71) Applicant: The General Hospital Corporation, Boston, MA (US)

(72) Inventors: Mikael J. Pittet, Charlestown, MA (US); Ralph Weissleder, Peabody, MA (US); Sean Arlauckas, Cambridge, MA (US); Christopher Garris, Wellesley, MA (US)

(21) Appl. No.: 16/495,725

(22) PCT Filed: Mar. 19, 2018
(86) PCT No.: PCT/US2018/023064

§ 371 Date: Sep. 19, 2019

Related U.S. Application Data
(60) Provisional application No. 62/562,583, filed on Sep. 25, 2017.
(60) Provisional application No. 62/484,649, filed on Apr. 12, 2017.
(60) Provisional application No. 62/473,734, filed on Mar. 20, 2017.

(51) Int. Cl. C07K 16/28; A61P 35/00; A61K 39/395; A61K 31/573

(52) U.S. Cl. CPC C07K 16/2818; C07K 16/2827; C07K 16/2878; C07K 16/283; A61K 2039/507; A61K 39/3955; A61K 31/573; C07K 2317/41; C07K 2317/52; A61P 35/00

(57) ABSTRACT

Fc-engineered variants of anti-PD1 IgG antibodies that abrogate FcγR binding and mAb effector functions, or combinations with therapies that inhibit FcγR binding in vivo, for treatment of cancer.

### CLAIM OF PRIORITY

This application claims the benefit of U.S. Provisional Patent Application Ser. Nos. 62/473,734, filed on Mar. 20, 2017; 62/484,649, filed on Apr. 12, 2017; and 62/562,583, filed on Sep. 25, 2017. The entire contents of the foregoing are hereby incorporated by reference.

### FEDERALLY SPONSORED RESEARCH OR DEVELOPMENT

This invention was made with Government support under Grant Nos. AI084880, CA164448, CA190344, CA151884, CA086355, AR068272 and HL084312 awarded by the National Institutes of Health. The Government has certain rights in the invention.

### TECHNICAL FIELD

Described herein are Fc-engineered variants of IgG antibodies, including anti-PD1, anti-CD40, and anti-PD-L1 IgG antibodies, that abrogate FcγR binding and mAb effector functions, or combinations with therapies that inhibit FcγR binding in vivo, for treatment of cancer.

### BACKGROUND

Immune checkpoint blockade is a recent development in cancer therapy that has shown remarkable results in certain cancers and patient groups (1-3). Currently approved immune checkpoint blockers are monoclonocal antibodies (mAbs) that target the programmed cell death protein 1 (PD-1) or cytotoxic T-lymphocyte-associated protein 4 (CTLA-4) pathways, and agents targeting other pathways are in clinical development (including OX40, Tim-3, and LAG-3) (4). Checkpoint inhibitors are used to reactivate exhausted tumor-specific T cells and reinstate cancer immuno-surveillance (5, 6). Indeed, some cancer tissues limit anti-tumor immunity by upregulating immunosuppressive factors such as PD-1 ligand (PD-L1) that binds to PD-1 on tumor-specific CD8⁺ T cells (7). Drugs targeting the PD-1/PD-L1 immune checkpoint axis can block immunosuppressive signals and enable T cell-mediated elimination of cancer cells (8). However, immune checkpoint blockade is not always effective, and we lack a complete understanding of the mechanisms that contribute to efficacy and resistance (9).

### SUMMARY

The present invention is based, at least in part, on the discovery that anti-PD1, anti-CD40, and anti-PD-L1 antibodies are quickly cleared from tumor cells by macrophages via an Fc-dependent mechanism. Thus, described herein are Fc-engineered variants of anti-PD1, anti-CD40, and anti-PD-L1 IgG antibodies that abrogate FcγR binding and mAb effector functions, or combinations with therapies that inhibit FcγR binding in vivo, for treatment of cancer.

Thus, provided herein are modified anti-PD1, anti-PD-L1, or anti-CD40 IgG antibodies with significantly reduced or abrogated Fc:FcγR binding interactions, wherein the antibodies have one or more of: (i) a modification to the primary sequence of Fc receptor to abrogate Fc receptor binding; (ii) removal of N-linked Glycosylation on Fc Portion of Antibody to abrogate Fc receptor binding; (iii) sialylation of Fc Portion of Antibody to abrogate Fc receptor binding; or (iv) altered glycosylation of Fc Portion of Antibody to abrogate Fc receptor binding.

In some embodiments, the antibody is a IgG1 with one or more of a mutation of Asparagine 297 to Alanine (N297A); mutation of Leucine 234 to Alanine and Leucine 235 to Alanine (LALA mtutation); mutation of Proline 329 to Glycine (P329G); and/or mutation of Leucine 235 to Glutamic Acid (L235E). In some embodiments, the antibody has a LALA mutation and mutation at P329G.

In some embodiments, the antibody is a IgG2 with one a mutation of Valine 234 to Alanine (V234A), Glycine 237 to Alanine (G237A), Proline 238 to Serine (P238S), Histidine 268 to Alanine (H268A), Valine 309 to Leucine, Alanine 330 to Serine (A330S), and Proline 331 to Serine (P331S) in the Fc Region (V234A/G237A/P238S/H268A/V309L/A330S/P331S).

In some embodiments, the antibody is a IgG1 with one or more of a mutation of Serine 228 to Proline and Leucine 235 to Glutamic Acid (L235E); mutation of Leucine 234 to Alanine and Leucine 235 to Alanine (LALA); and/or mutation of Serine 228 to Proline and Leucine 235 to Glutamic Acid (L235E) and Proline 329 to Glycine (P329G).

In some embodiments, N-linked Glycosylation on Fc Portion of Antibody was removed by digestion of N-linked glycan using Peptide:N-Glycosidase F (PNGase F).

In some embodiments, the antibody is an IgG1 that has been sialylated using chemoenzymatic glycosylation remodeling.

In some embodiments, an IgG4 that exclusively contains GOF glycans at N297.

In some embodiments, the antibody is a modified anti-PD1 antibody, preferably selected from the group consisting of pembrolizumab, nivolumab, avelumab, pidilizumab, and atezolizumab; a modified anti-CD40 antibody, preferably selected from the group consisting of dacetuzumab, lucatumumab, bleselumab, teneliximab, ADC-1013, CP-870,893, Chi Lob 7/4, HCD122, SGN-4, SEA-CD40, BMS-986004, and APX005M; or an anti-PD-L1 antibody, preferably selected from the group consisting of BMS-936559, FAZ053, KN035, Atezolizumab, Avelumab, and Durvalumab.

Also provided herein are pharmacological compositions comprising the modified antibodies described herein.

Further provided herein are methods for treating a cancer in a subject comprising administering a therapeutically effective amount of a modified antibody as described herein to a subject in need thereof.

In addition, provided herein are compositions comprising an anti-PD1, anti-PD-L1, or anti-CD40 antibody and an anti-Fc gamma receptor antibody.

Also provided herein are methods for treating a cancer in a subject comprising administering a therapeutically effective amount of an anti-PD1, anti-PD-L1, or anti-CD40 antibody and an anti-Fc gamma receptor antibody, preferably Fc gamma specific antibodies that engage activating Fc gamma receptors. In some embodiments, the anti-Fc receptor antibody binds Fc gamma receptors I, II, and III. In some embodiments, the anti-Fc receptor antibody is selected from the group consisting of antibodies that bind specifically to human Fc gamma receptor IIb, and antibodies that bind specifically to human Fc gamma receptor IIb.

Also provided herein are compositions comprising an anti-PD1 anti-PD-L1, or anti-CD40 antibody and prednisolone.

Further, provided herein are methods for treating a cancer in a subject comprising administering a therapeutically effective amount of an anti-PD1 anti-PD-L1, or anti-CD40 antibody and prednisolone, or a therapeutically effective amount of an anti-PD1 anti-PD-L1, or anti-CD40 antibody and an agent that reduces levels of tumor Fc gamma receptor expressing macrophages selected from small molecule or antibody inhibitors of CSF1 and small interfering RNA (siRNA) directed against CCR2.

In addition, provided herein are the antibodies and compositions described herein for use in a method of treating a subject who has cancer.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and Figures, and from the claims.

### DESCRIPTION OF DRAWINGS

FIGS. 1A-1D. Anti-PD-1 mAb labeling facilitates tracking of tissue biodistribution. (1A) The rat anti-mouse PD-1 29F.1A12 clone, conjugated to Alexa Fluor 647 via NHS ester linkage, efficiently binds PD-1+ T cells (here EL4 cells) as detected by flow cytometry (gray histogram). Isotype control staining is shown in white. (1B) MC38 tumors were equally responsive to single-dose AF647-aPD-1 and unlabeled aPD-1, whereas tumor sizes increased 72 h after control IgG2a treatment. (1C) Fluorescence reflectance imaging of 3 tumors compared to tumor draining (tdLN) and non-draining (ndLN) lymph nodes 24 hours after AF647-aPD-1 treatment (AF647: λₑₓ=620-650 nm, λₑₘ=680-710 nm). Scale bars represent 5 mm. (1D) Quantified AF647-aPD-1 in each tissue demonstrating tumor accumulation. Values represent SEM and n=3 unless otherwise noted. ***P<0.001; unpaired, two-tailed t-test. A-B: Specificity of aPD-1 mAb is preserved after AF647 labeling FIGS. 2A-2C: In vivo temporal aPD-1 mAb pharmacokinetics reveals drug accumulation in TAMs. (2A) Diagram depicting intravital imaging setup with labeled aPD-1, MC38 tumor cells, T cells, and tumor associated macrophages (TAMs). (2B) Treatment with single-dose aPD-1 mAb can achieve remission in the MC38/H2B-mApple tumor model. Tumors are outlined in gray; scale bars represent 2 mm. (2C) Intravital microscopy (IVM) biodistribution studies indicate early aPD-1 binding to T cells and long-term accumulation in TAMs. Data are representative of 5 independently-treated DPE-GFP mice and normalized to autofluorescent signal.

FIGS. 3A-3D: In vivo imaging reveals aPD-1 mAb transfer from CD8⁺ T cells to TAMs. (3A) Quantified AF647 signal on T cells and TAMs from a representative DPE-GFP mouse demonstrates collection of AF647-aPD-1 in T cells at 15 minutes and in TAMs at 24 hours after injection of therapy. (3B) Quantification of aPD-1-mAb on IFNγ-expressing CD8⁺ lymphocytes and TAMs reveals a narrow window of target binding. (3C) Flow cytometry histograms pre-gated for 7-AAD⁻/CD45⁺ show AF647-aPD-1 signal (x-axis, logarithmic scale) on immune cell populations at 0.5 hr and 24 hours after administration. Cell populations from untreated control animals were used as reference. (3D) aPD-1 mAb binds to CD8⁺ lymphocytes early but accumulates in TAMs at later time points. **P<0.01; ****P<0.0001; unpaired, two-tailed t-test.

FIGS. 4A-4D: aPD-1 mAb transfer to macrophages is mediated by FcγRs. (4A) Ex vivo flow cytometry histograms of MC38 tumors stained with PE-aPD-1 show that CD8⁺ T cells but not TAMs express cell surface PD-1. (4B) Co-culture of bone marrow-derived macrophages (Mo) and AF647-aPD-1 coated EL4 lymphocytes was used to quantify the AF647-aPD-1 puncta in macrophages pre-blocked with FcγR inhibitors or the phagocytosis inhibitor, dynasore. *P<0.05; ****P<0.0001; one-way ANOVA. (4C-4D) Flow cytometry was used to estimate AF647-aPD-1 transfer to F4/80⁺ BMDMs in co-culture assays when the mAb was added directly, bound to EL4 cells, or bound to EL4 cells in the presence of FcγRII/III neutralizing antibody. Results in 4C are a representative histogram of AF647 signal in macrophages and 4D is G-MFI value of conditions presented in 4C. Data are from 3 independent experiments. *P<0.05; ** P<0.01; two-way ANOVA with Tukey's multiple comparisons test. Values represent SEM for three separate experiments.

FIGS. 5A-5B: Nivolumab shares similar glycan patterns with mouse aPD-1 and is transferred to macrophages via FcγRs. (5A) HPLC analysis of the glycan patterns found on the mouse aPD-1 mAb and nivolumab shows the GOF (“2”) isoform to be predominant, but glycosylation is not uniform. (5B) AF647-labeled nivolumab was used to stain the surface of aCD3 stimulated PKH-green labeled human CD8⁺ T cells co-incubated with PKH-red labeled peripheral blood mononuclear cell-derived macrophages in the presence or absence of Fc Block. Quantification of AF647⁺ puncta per macrophage confirms that nivolumab is transferred via FcγRs. Values represent SEM for 4 separate experiments.

FIGS. 6A-6D: Disrupting Fc binding affects macrophage uptake of aPD-1 and improves treatment efficacy. (6A) PKH-green labeled EL4 cells were stained with native AF647-aPD-1 or deglycosylated AF647-aPD-1 co-cultured with PKH-red labeled bone marrow-derived macrophages (Mo). FACS plots of mouse Mø (gated on F4/80⁺) co-cultured with PD-1+EL4 cells labeled with either AF647-aPD-1 mAb or deglycosylated AF647-aPD-1 mAb. Asterisks are used for identification in this Figure and do not indicate significance. (6B) aPD-1-mAb deglycosylation substantially reduces the transfer from EL4 cells to Mø (n=3). **P<0.01; Unpaired 2-tailed t test. (6C) Quantified AF647-aPD-1 from three 2.4G2-treated mice shows prolonged aPD-1 binding to tumor T cells, relative to mice treated with AF647 aPD-1 alone (data repeated from FIG. 3B for comparison). (6D) MC38 tumor growth curves of mice (n>5) treated with isotype control, aPD-1, and aPD-1 plus 2.4G2. *P<0.05 One-Way ANOVA with Tukey's multiple comparison test.

FIG. 7: AF647-aPD-1 mAb quantification in various tissues at 24 h injection. Fluorescence reflectance imaging (left) was used to detect AF647-aPD-1 mAb within various tissues, which were resected from MC38 tumor-bearing mice at 24 h post drug administration (AF647: λₑₓ=620-650 nm, λₑₘ=680-710 nm). Bright-field images of the same field of views are also shown (right). Scale bar represents 5 mm. ndLN: non-draining lymph node; tdLN: tumor-draining LN.

FIGS. 8A-8C: Specificity of T cell reporter mice and dextran nanoparticles. (8A) Representative flow cytometry plot of tumor-infiltrating cells obtained from a MC38 tumor-bearing DPE-GFP mouse, indicating that DPE-GFP+ cells are also CD90+(T cell marker). (8B) IFNγ reporter expression profiles of various tumor-infiltrating immune cell populations obtained from a MC38 tumor-bearing IFNγ reporter (GREAT) mouse. Cell populations shown are: CD8⁺ T cells (pre-gated as CD3+CD8+), macrophages (pre-gated as F4/80+), and remaining F4/80− CD8− cells. (8C) Representative flow cytometry dot plot of MC38 tumor-bearing mice injected 24 hours prior with Pacific Blue-dextran nanoparticle shows uptake specific to F4/80+ cells pre-gated for CD45. Asterisks are used for identification in this Figure and do not indicate significance.

FIGS. 9A-9D: T cell and macrophage motility before and after aPD-1 treatment. (9A) Track plots of GREAT mouse T cells imaged using intravital microscopy. (9B) Motility coefficients of individual T cell tracks were not significantly different after aPD-1 injection. (9C) Instantaneous T cell velocity did not change after treatment. (9D) Tumor macrophage motility coefficients showed the cells to be stationary before and after aPD-1 injection. Pre-treatment refers to a 20-minute period before aPD-1 injection, and post-treatment refers to a 20-minute period after aPD-1 injection.

FIG. 10: Assessment of aPD-1 binding to tumor cells. In vivo microscopy in the dorsal skin fold chamber of a GREAT mouse with MC38/H2B-mApple tumor cells revealed no evidence of AF647-aPD-1 binding to tumor cells at any of the times investigated Lack of AF647-aPD-1 binding to tumor cells was consistent with flow cytometry histograms demonstrating no PD-1 expression in MC38 tumor cells. Stained and unstained EL4 cells were used as positive and negative control samples.

FIG. 11: aPD-1 transfer to tumor macrophages in vivo. Mice bearing MC38 tumors were sacrificed 2 h after receiving aPD-1. Single cell suspensions from tumors were fixed, permeabilized, and stained for multicolor flow cytometry to identify aPD-1 (Rat IgG2a) in tumor macrophages (CD45+ CD11b+ MHCII+), confirming that the antibody is transferred in vivo.

FIGS. 12A-12C: Distribution of AF647-aPD-1 across tumor models. Mice bearing the indicated tumors were sacrificed 24 h after receiving AF647-aPD-1. (12A) Multicolor flow cytometry was performed ex vivo to identify AF647-aPD-1 cellular distribution among immune cell types, including macrophages (CD45+ 7AAD− F4/80+ CD11c+), T cells (CD45+ 7AAD− CD90+CD11b−), CD45+ 7AAD− CD11b+F4/80− cells that include granulocytes/monocytes, and other immune cells (CD45+ 7AAD− CD90− CD11b− F4/80− CD11c−) Data are averages of 4 independent samples. (12B) Percentage of tumor-associated macrophages accumulating AF647-aPD-1. n=4 for each tumor type. (12C) AF647-aPD-1 binding ability to tumor-associated macrophages across a range of tumor-associated macrophage densities (Triangles: MC38, Circles: B16, Squares: KP1.9). The graph shows the percent of anti-PD-1 mAbs bound to macrophages (y-axis; defined as the fraction of AF647+ signal bound to these cells) in tumors with varying percentages of these cells (x-axis; defined as the fraction of F4/80+ CD11c+ cells among CD45+ 7AAD− cells). Linear regression analysis of combined data indicates a non-significant (n.s.) p value for slope deviation from zero. Each datapoint identifies a different tumor.

FIG. 13: PD-1 expression by CD8⁺ T cells in the MC38 tumor microenvironment. A representative flow cytometry histogram shows PD-1 expression on CD8+ T cells, NK Cells, and B cells sorted from MC38 tumors implanted intradermally in C57B6 mice. Pooled results from four tumors show substantially higher surface PD-1 expression on tumor infiltrating CD8+ T cells than the negligible amount found on other immune cell types. *P<0.05; two-way ANOVA with Tukey's multiple comparisons test.

FIG. 14. aPD-1 mAb transfer from T cells to macrophages in vitro. AF647-aPD-1 transfer from T cells (EL-4 cells) to macrophages was observed in 3 independent experiments run in parallel with an AF647-labeled control rat IgG2a. The number of AF647+ puncta in macrophages was quantified for the AF647-labeled aPD-1, AF647-labeled control rat IgG2a, or AF647-labeled PNGaseF-treated aPD-1 mAbs. Values are reported relative to the AF647-aPD-1 condition. ****P<0.0001; one-way ANOVA. Scale bars represent 10 μm.

FIGS. 15A-15B: Assessment of aPD-1 internalization after binding to PD-1. (15A) An experimental scheme using AF647-aPD-1 stained EL4 lymphocytes before or after 1 hour incubation at 37 degrees Celsius provides a model to study antibody internalization. Acid wash removal of surface antibody permits detection of internalized aPD-1 using fluorescence measurements. (15B) Flow cytometry measurement of AF647-aPD-1 levels in freshly-stained T lymphocytes dropped several orders of magnitude after acid wash removal of surface antibody. Acid stripped lymphocytes incubated at 37 degrees for 1 hour showed negligible levels of internalized AF647-aPD-1. Data represent 2 independent experiments.

FIGS. 16A-B: aPD-1 degradation by macrophages. (16A) Experimental outline of aPD-1 coated T cell macrophage co-culture conditions. (16B) ELISA-based detection of rat IgG2a in bone marrow-derived macrophages (Mo) or supernatants after incubation periods (2 and 6 h) following removal of aPD-1-labeled lymphocytes. Antibody detected in macrophages after supernatant replacement was significantly reduced after 4 additional hours of culturing. Antibody release into the supernatant was not detected (n=3). *P<0.05; t test.

FIGS. 17A-17B: PD-1 localization after aPD-1 internalization after binding to PD-1. (17A) To determine PD-1 receptor fate after aPD-1 mAb transfer, T cells coated with unlabeled aPD-1 and co-cultured with macrophages (Mo) to permit antibody removal. PD-1 receptor remaining on the T cell surface after antibody transfer is detected by re-staining with PE-aPD-1 after acid washing. Flow cytometric analysis showed Mø removal of aPD-1 permitted binding of PE-aPD-1 to the exposed surface PD-1 on EL4 lymphocytes (17B). Following acid wash, PE-aPD-1 levels were high on lymphocytes regardless of Mø exposure. Data represent 2 independent experiments; *P<0.05; **P<0.01; two-way ANOVA with Tukey's multiple comparisons test.

FIGS. 18A-18B. Comparative analysis of mAb glycosylation patterns between mouse and human PD-1 antibodies. (18A) Total glycan was digested from mAb using recombinant PNGase F. (18B) HPLC analysis of glycan digested from nivolumab (top) or rat IgG2a anti-mouse PD-1 (29F.1A12 clone), and the distribution of glycan structures based on size exclusion. The y-axis is arbitrary units indicative of glycoform abundance, and x-axis is time of elution from the column. The digested glycans from human IVIG (not shown) were used as a reference to label the elution times for common glycoforms.

FIGS. 19A-19B. Confirmation of deglycosylation and antigen binding affinity for rat anti-mouse PD-1. (19A) Rat anti-mouse PD-1 mAb (clone 29F.1A12) and rat IgG2a isotype control were deglycosylated using PNGase F, and lens culinaris agglutinin (LCA) agent for visualizing sugars was used to confirm complete deglycosylation. (19B) PNGase F treated aPD-1 conjugated to AF647 and was used to label PD-1 expressing cells (filled histogram). The open histogram is rat IgG2a isotype control staining.

FIG. 20: Impact of Fc blockade on aPD-1 treatment efficacy. MC38 tumor growth curves for individual mice treated with isotype control, aPD-1, or aPD-1 plus 2.4G2 demonstrate the heterogeneity in aPD-1 response and subsequent increase in complete regressions when Fc binding is abrogated.

FIG. 21: Proposed resistance mechanism and potential improvement of aPD-1 mAb therapy. The left panel shows a model in which tumor-associated macrophages (TAM) limit aPD-1 mAb engagement of T cell PD-1 receptors by sequestering the drug using Fcγ receptors (FcγR). PD-1 binding to its ligand (PD-L1 expressed by tumor cells) inhibits anti-tumor T cell immunity. The right panel shows a model in which FcγR blockade results in aPD-1 retention on T cells. This is turn augments anti-tumor functions and reverses the heterogeneity observed in aPD-1 mAb treatment response.

FIGS. 22A-22B: Antibody transfer is not solely dependent on the inhibitory FcγRIIb receptor. Macrophages derived from the bone marrow of wild-type (WT) or FcgR2b knockout (KO) C57B6 mice were labeled with PKH-red and co-cultured with T cells stained with PKH-green and AF647-aPD-1. Time lapse microscopy showed aPD-1 was still removed by FcgR2b KO macrophages. (22A) Independent analysis by flow cytometry confirmed the antibody transfer mechanism was blocked by CD16/CD32 blockade (Fc Block) but not FcgR2b knockout alone. (22B) Flow cytometric values are pooled across independent experiments.

FIGS. 23A-23B: Not all Fc-modifying strategies yield effective in vivo aPD-1 therapies. (23A) The rat anti-mouse PD-1 (left) and isotype control (right) were treated with Endo S Agarose beads (lanes 2 and 4) and compared to the native antibody (lanes 1 and 3). Coomassie staining following SDS-PAGE confirmed the shift in immunoglobulin size consistent with Endo S deglycosylation (top). Secondary confirmation of Endo S deglycosylated in lanes 2 and 4 was demonstrated using lens culinaris agglutinin (LCA) agent for lectin blotting (bottom). (23B) Deglycosylation of aPD-1 using Endo S strategies does not improve survival in mice bearing MC38 colorectal tumors. These data provide added insight by eliminating obvious strategies that provide no therapeutic benefit.

FIG. 24: Flow cytometry data quantifying anti-PD-1 uptake into macrophages. Fluorescence-activated cell sorting plots of mouse Mø (gated on F4/80+) cocultured with PD-1+EL4 cells labeled with either AF647-aPD-1 mAb or deglycosylated AF647-aPD-1 mAb. aPD-1 mAb deglycosylation substantially reduces the transfer from EL4 cells to Mø (n=3). **P<0.01, unpaired two-tailed t test.

FIG. 25: Prednisolone dampens activating FcγR mRNA expression. Analysis by RT-qPCR shows the expression of FcγR1 and FcγR3A to dramatically decrease during 7 day exposure to prednisolone, the active form of prednisone. The low affinity Fc receptors (FcγR2A and FcγR2B) did not dramatically change during this time. These data are representative of two healthy volunteers from which peripheral blood multinuclear cells were differentiated into macrophages.

### DETAILED DESCRIPTION

At present, experimental and clinical evidence suggest that a pre-existing tumor infiltrate of CD8⁺ T cells is one of the most favorable prognostic indicators of checkpoint inhibitor response (10). Also, patients with the highest degree of neoantigen burden (high mutational load) in their cancers may have increased tumor infiltration by T cells and more robust responses to checkpoint blockade (11,12). Histology and sequencing methodologies have been used to define metrics of cytotoxic T cell infiltration in tumors (13), with a focus on the identification of tumor neoantigens and the resultant antigen-specific T cell expansion after immunotherapy (11). These studies have provided insight into the mechanism of aPD-1 mAb-induced antitumor T cell activation and spurred efforts focused on identifying new strategies that foster T cell recruitment to tumors (14-16).

Much less is known about checkpoint inhibitors' in vivo pharmacokinetics and interactions with host components in the tumor bed. Studying these parameters is likely essential to identifying resistance mechanisms and developing improved therapeutic options. Herein, intravital imaging was used to follow fluorescently-labeled aPD-1 mAbs in real-time and at subcellular resolution. Because tumor microenvironments are home to diverse host cell types, and immune checkpoint blockers are unlikely to solely act on T cells, the study focused on aPD-1 mAb interactions with various host components by simultaneously assessing aPD-1 mAbs, tumor cells, CD8 T cells, and myeloid cells/macrophages. Tumor-infiltrating CD8⁺ T cells were investigated because they express PD-1 and are the expected targets of aPD-1 mAbs. Myeloid cells were also investigated because they are frequently found in the stroma of growing tumors (17) and emerging evidence indicates that they can affect virtually all therapeutic modalities, including immunotherapy (18). The results confirmed existing knowledge of PD-1 inhibition mechanisms, but also uncovered findings with therapeutic implications to further improve immunotherapy.

Many cancer patients do not respond to immune checkpoint blockade therapy, and there is an incomplete understanding of the mechanisms that contribute to treatment efficacy and resistance. Herein, time-lapse intravital microscopy was used to uncover in real-time how the immune checkpoint blocker aPD-1 mAb distributes in tumors and physically interacts with tumor microenvironment components. This approach enabled the detection of the association of aPD-1 mAb with cytotoxic T cells infiltrating tumors in vivo. Furthermore, by following the drug's pharmacokinetics over time, the drug was found to be rapidly removed from PD-1⁺ CD8⁺ T cells and transferred to neighboring PD-1⁻ tumor-associated macrophages. The transfer of aPD-1 mAbs from T cells to macrophages was unexpected because macrophages do not directly take up aPD-1 mAbs in culture. It was further determined that aPD-1 uptake by macrophages depends both on the Fc domain of the antibody and on FcγRs expressed by macrophages. Interactions between the drugs and macrophages are likely important, because blocking Fc:FcγR binding inhibited aPD-1 transfer from CD8⁺ T cells to macrophages in vivo and enhanced aPD-1 therapeutic efficacy.

Although clinical aPD-1 has an extensive half-life in the circulation (˜26 days), the present observations suggest that the time of target engagement in the local tumor environment may be much shorter. This engagement time is reduced at least in part by FcγRII/III, which mediate aPD-1 mAb uptake from T cells to macrophages. Accordingly, previous work in mice has shown that IgG2a isotypes preferentially bind FcγRIIb/III and that aPD-1 therapy with an IgG2a mAb is more effective in FcγRIIb knockout animals (25). The present studies further suggest that FcγR-mediated aPD-1 removal does not involve transfer of cell membrane components, or trogocytosis, which has been described for other mAbs including rituximab (27, 28). However, as shown herein, aPD-1 uptake by macrophages is favored when the mAb is bound to PD-1 on T cells, which aligns with previous findings that FcγRs bind IgGs more efficiently when they form immune complexes (29, 30). It was also found that PD-1 remains on the T cell surface after aPD-1 removal, but these PD-1 molecules did not bind new aPD-1. Overall, aPD-1 transfer from T cells to macrophages appears to be faster than aPD-1 uptake by T cells in the tumor stoma.

The human aPD-1 drugs nivolumab and pembrolizumab were designed as human IgG4 antibody isotypes that are not known to fix complement or trigger ADCC (31). However, IgG4 can bind FcγRI and FcγRIIb, and these interactions can have profound clinical consequences (32, 33). Awareness of the FcγR binding profile of a mAb offers an opportunity to improve upon existing monoclonal therapies, exemplified by obinutuzumab, a de-fucosylated IgG1 bio-similar of rituximab designed to bind FcγRIIIA and enhance ADCC against CD20+ cells in chronic lymphocytic leukemia (34). Human germ-line variants of FcγRs that display altered Fc binding tropism have been identified and are an important focus in the effort to understand responses to mAb therapies that rely on FcγRs for therapeutic function, like cetuximab (35), rituximab (36), trastuzumab (37), and other mAb therapies (38).

Without wishing to be bound by theory, it is suggested that nivolumab and other IgG4-based mAbs are not exceptions to the rules of FcγR binding and therefore Fc interactions should be considered in pharmacologic models. This is particularly important since there is growing interest in immune checkpoint molecules as diagnostic tools to identify PD-1/PD-L1⁺ tumors. For example, prior efforts to image PD-1 expression using PET radio-ligands have focused on gross tissue distribution and lack the resolution to identify cellular tropisms in vivo. Natarajan et. al. used hamster anti-mouse PD-1 (39), but it is not fully understood how hamster mAbs interact with mouse FcγRs in this context. A secondary aPD-1 PET imaging study reported the use of the RMP1-14 rat IgG2a anti-mouse PD-1 clone to cross-correlate with ex vivo PD-1 staining (40), but drug withdrawal by macrophages could complicate the relationship between PET signal and PD-1 expression. Future pre-clinical diagnostic efforts to image PD-1 expression should consider imaging agents that avoid FcγR interactions; however, antibodies meant to mimic nivolumab and pembrolizumab should accurately represent the Fc status of the human IgG4 antibodies.

The findings from the present study support new methods of improving current treatment options as described herein. Based on this understanding of Fc interactions with aPD-1, Fc-engineered IgG variants that abrogate FcγR binding and mAb effector functions (41), or combinations with therapies that inhibit FcγR binding in vivo, are used enhance the effects of treatment.

Methods of Treatment

The methods described herein include methods for the treatment of cancer, e.g., solid tumors. Generally, the methods include administering a therapeutically effective amount of a modified antibody or combination therapy as described herein, to a subject who is in need of, or who has been determined to be in need of, such treatment.

As used in this context, to “treat” means to ameliorate at least one symptom of the disorder associated with the cancer. Administration of a therapeutically effective amount of a compound described herein for the treatment of a cancer can result in, for example, one or more of decreased tumor size; decreased tumor growth rate; decreased risk of metastasis; and/or decrease risk of reoccurrence.

As used herein, the terms “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth.

The terms “cancer” or “neoplasms” include malignancies of the various organ systems, such as affecting lung, breast, thyroid, lymphoid, gastrointestinal, and genito-urinary tract, as well as adenocarcinomas that include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CIVIL) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

The present methods can be used in any cancer in which the anti-PD-1, anti-CD40, or anti-PD-L1 antibody has therapeutic effect.

### Modified Antibodies

Thus, provided herein are compositions that comprise anti-PD-1, anti-CD40, and anti-PD-L1 antibodies with significantly reduced or abrogated Fc:FcγR binding interactions, and methods of use thereof. The antibodies are monoclonal antibodies and can be, e.g., recombinant, chimeric, de-immunized or humanized, or fully human. The antibodies include an Fc region (preferably an Fc region from IgG1, IgG2, or IgG4) linked via a hinge region to an antigen binding region, e.g., at least one Fab domain that binds specifically to PD-1, CD40, or PD-L1.

Exemplary anti-PD-1 antibodies that can be used in the methods described herein include those that bind to human PD-1; an exemplary PD-1 protein sequence is provided at NCBI Accession No. NP_005009.2. Exemplary antibodies are described in U.S. Pat. Nos. 8,008,449; 9,073,994; and US20110271358, including PF-06801591, AMP-224, BGB-A317, BI 754091, JS001, MEDI0680, PDR001, REGN2810, SHR-1210, TSR-042, pembrolizumab, nivolumab, avelumab, pidilizumab, and atezolizumab.

Exemplary anti-CD40 antibodies that can be used in the methods described herein include those that bind to human CD40; exemplary CD40 protein precursor sequences are provided at NCBI Accession No. NP_001241.1, NP_690593.1, NP_001309351.1, NP_001309350.1 and NP_001289682.1. Exemplary antibodies include those described in WO2002/088186; WO2007/124299; WO2011/123489; WO2012/149356; WO2012/111762; WO2014/070934; US20130011405; US20070148163; US20040120948; US20030165499; U.S. Pat. No. 8,591,900; including dacetuzumab, lucatumumab, bleselumab, teneliximab, ADC-1013, CP-870,893, Chi Lob 7/4, HCD122, SGN-4, SEA-CD40, BMS-986004, and APX005M. In some embodiments, the anti-CD40 antibody is a CD40 agonist, and not a CD40 antagonist.

Exemplary anti-PD-L1 antibodies that can be used in the methods described herein include those that bind to human PD-L1; exemplary PD-L1 protein sequences are provided at NCBI Accession No. NP_001254635.1, NP_001300958.1, and NP_054862.1. Exemplary antibodies are described in US20170058033; WO2016/061142A1; WO2016/007235A1; WO2014/195852A1; and WO2013/079174A1, including BMS-936559 (MDX-1105), FAZ053, KN035, Atezolizumab (Tecentriq, MPDL3280A), Avelumab (Bavencio), and Durvalumab (Imfinzi, MEDI-4736).

These modified antibodies can be created in a number of ways, including:

a) Modification to the Primary Sequence of Fc Receptor to Abrogate Fc Receptor Binding

Methods to abrogate Fc:Fc receptor binding encompass mutational changes to the primary coding sequence of the given Fc portion of an antibody. This can vary depending upon Fc isotype, and the following list provides exemplary strategies for each specific antibody class.

IgG1—(wild type sequence Genbank Acc. No. AF237583.1) Mutation of Asparagine 297 to Alanine (N297A); Mutation of Leucine 234 to Alanine and Leucine 235 to Alanine (LALA); Mutation of Proline 329 to Glycine (P329G); Mutation of Leucine 235 to Glutamic Acid (L235E); Combination of LALA mutation and P329G. See, e.g., Schlothaouer et al., Protein Eng Des Sel. 2016 October; 29(10):457-466.

IgG2—(wild type sequence Genbank Acc. No. AF237584.1) Mutation of Valine 234 to Alanine (V234A), Mutation of Glycine 237 to Alanine (G237A), Proline 238 to Serine (P238S), Histidine 268 to Alanine (H268A), Valine 309 to Leucine, Alanine 330 to Serine (A330S), and Proline 331 to Serine (P331S) in the Fc Region, i.e., V234A/G237A/P238S/H268A/V309L/A330S/P331S. See Methods. 2014 Jan. 1; 65(1):114-26.

IgG4—(wild type sequence Genbank Acc. No. AF237586.1) Combination mutation of Serine 228 to Proline and Leucine 235 to Glutamic Acid (L235E); Mutation of Leucine 234 to Alanine and Leucine 235 to Alanine (LALA); Combination mutation of Serine 228 to Proline and Leucine 235 to Glutamic Acid (L235E) and Proline 329 to Glycine (P329G). See, e.g., Schlothaouer et al., Protein Eng Des Sel. 2016 October; 29(10):457-466.

See also Reusch and Tejada et al., Glycobiology. 2015 December; 25(12): 1325-1334.

b) Removal of N-Linked Glycosylation on Fc Portion of Antibody

N-linked glycans are essential for Fc gamma to Fc gamma receptor binding. Methods to remove them can be performed through a variety of enzymatic protocols. This encompasses digestion of N-linked glycan using Peptide:N-Glycosidase F (PNGase F), which cleaves total glycan from the protein. In some embodiments, it also includes digestion of antibody with EndoS enzyme, which specifically removes glycan from the Fc portion of the antibody.

As shown in Examples 3 and 4, below, digestion of Fc glycan using PNGase F blocks antibody uptake by macrophages.

c) Sialylation of Fc Portion of Antibody

Sialylated IgG1 drugs have minimal FcR engagement. In some embodiments, chemoenzymatic glycosylation remodeling can be used to add sialic acid to the Fc, e.g., 4 sialic acids per Fc fragment as described in Huang et al. J Am Chem Soc. 2012 Jul. 25; 134(29):12308-18 and Quast et al., J Clin Invest. 2015 Nov. 2; 125(11): 4160-4170. Other methods include (1) treatment of antibody with recombinant sialyltransferase enzymes (e.g., as described in Hidari et al., Glycoconj J. 2005 February; 22(1-2):1-11 and Thomann et al., PLoS One. 2015 Aug. 12; 10(8):e0134949), (2) overexpression of sialyltransferase genes in antibody production cells (e.g., as described in Onitsuka et al., Appl Microbiol Biotechnol. 2012 April; 94(1):69-80 and Raymond et al., MAbs. 2015 May-June; 7(3): 571-583), (3) modification of the culture conditions of antibody-producing cells to enhance production of sialylated antibodies (e.g., as described in Jones et al., Proc Natl Acad Sci USA. 2016 Jun. 28; 113(26):7207-12 and Andersen and Goochee, Curr Opin Biotechnol. 1994 October; 5(5):546-9).

d) Altered Glycosylation of Fc Portion of Antibody

Human IgGs, e.g., IgG4s, can be prepared to exclusively contain GOF glycans at N297. See, e.g., Jefferis, “Posttranslational Modifications and the Immunogenicity of Biotherapeutics,” Journal of Immunology Research, vol. 2016, Article ID 5358272, 15 pages, 2016; Li et al., Proc Natl Acad Sci USA. 2017 Mar. 28; 114(13):3485-3490; and Dekkers et al., Scientific Reports 6, Article number: 36964 (2016).

### Combination Therapy to Block Fc:Fc Receptor Binding

As an alternative to the modified antibodies described above, the methods can include a combination therapy including blockade of Fc gamma receptors administered with checkpoint immunotherapy.

As shown herein, co-administration of Fc receptor blocking antibodies with anti-PD-1 enhances immunotherapy efficacy; see Example 4, below. Thus, the present methods can include co-administration of anti-PD-1 with Fc receptor-specific antibodies that bind Fc gamma receptors I/II/III. For example, this can be achieved with Clone 2.4G2 that binds to murine Fc gamma receptors. Specific elimination of Fc gamma receptor expressing cells can be performed using Fc gamma specific antibodies that engage activating Fc receptors. These antibodies can include AT10, 7C07, 5C04, or 5C05, which are specific to human Fc gamma receptor IIb (see Roghanian et al., 2015. Cancer cell, 27(4):473-488) and the 5A6 antibody, which is specific to human Fc gamma receptor IIb (see U.S. Pat. No. 7,662,926B2; Jackman et al., 2010. Journal of Biological Chemistry, 285(27):20850-20859).

Co-administration can include administration of the agents concurrently (e.g., co-infusion of a single composition comprising both the anti-PD-1 and Fc receptor-specific antibodies), or subsequent administration, e.g., administration of the second within 6, 4, 2, or 1 hour, within 45, 30, 20, 15, 10, 5 or 2 minutes of the first. Preferably, the Fc-Receptor specific antibody is administered first.

### Combination Therapy to Reduce Fc Receptor Expression Levels

Additional immune-modulating agents that can alter the expression profile of human FcγRs offer a further strategy to modulate the receptors responsible for limiting aPD-1 efficacy. As shown in example 8, prednisolone (an anti-inflammatory glucocorticoid) diminished the surface expression of FcγRs; in particular, expression of CD64 (FcγR1) and CD16 (FcγR3A) dramatically decreased during a 1 week time-course of prednisolone. The low affinity CD32 Fc receptors (FcγR2A and FcγR2B) did not detectably change during this time-course. These data indicate a treatment that “primes” FcγRs for maximal aPD-1 efficacy. Thus, the methods can include co-administration of anti-PD-1 with agents that deplete surface Fc gamma receptors such as prednisolone and other corticosteroids. Co-administration can include administration of the agents concurrently (e.g., co-infusion of a single composition comprising both the anti-PD-1 and prednisolone), or subsequent administration, e.g., administration of the second within 6, 4, 2, or 1 hour, within 45, 30, 20, 15, 10, 5 or 2 minutes of the first. Preferably, the prednisolone is administered first. In some embodiments, another corticosteroid other than prednisolone is used, e.g., cortisone, hydrocortisone or prednisone.

### Combination Therapy to Reduce Levels of Tumor Fc Gamma Receptor Expressing Cells

Macrophages express significant amounts of Fc gamma receptors (Fcgr1, Fcgr2a, Fcgr2b, Fcgr3a) in the tumor microenvironment. Depleting macrophages can be used to prevent therapeutic antibody uptake. Depletion of tumor macrophages can be accomplished by several methods. First, targeting of the colony stimulating factor one (CSF-1) signaling axis can reduce macrophage within the tumor microenvironment. Second, inhibition of macrophage precursor recruitment to the tumor site can hinder macrophage development in the tumor microenvironment. This can be accomplished using agents that block CCR2 (e.g., small interfering RNA (siRNA) or other inhibitory nucleic acids directed against CCR2), which is an essential signal for recruitment of macrophage precursors to the tumor microenvironment. Other agents depleting macrophages, such as small molecule or antibody based inhibitors of colony stimulating factor 1 (CSF1) signaling and/or FcgR+ cells are equally relevant.

Therapies designed to target tumor macrophages (18), when combined with aPD-1, may provide additional benefit by increasing immune checkpoint blockade drug delivery to CD8⁺ T cells, thereby enhancing activity of immunotherapy. Clinical trials combining macrophage targeting therapeutics and immune checkpoint blockers are underway (42). In support of the notion that myeloid cells interface with aPD-1 immunotherapy, recently identified correlates of aPD-1 response in tumors suggest that alterations in macrophage gene signatures are associated with non-responsiveness to aPD-1 (43).

Pharmaceutical Compositions and Methods of Administration

The methods described herein include the use of pharmaceutical compositions comprising the modified antibodies or combination agents as active ingredients.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Pharmaceutical compositions are typically formulated to be compatible with its intended route of administration. Examples of routes of administration include parenteral, e.g., intravenous, intradermal, or subcutaneous; and oral administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### Examples

The invention is further described in the following examples, which do not limit the scope of the invention described in the claims.

Materials and Methods

The following materials and methods were used in the Examples below.

Materials

Rat IgG2a kappa anti-mouse PD-1 29F.1A12 clone was kindly provided by Gordon Freeman (DFCI). The SAIVI Alexa Fluor 647 Antibody/Protein 1 mg-Labeling Kit (ThermoFisher Scientific) was used to label Rat IgG2a isotype control clone 2A3 (BioXcell), rat anti-mouse PD-1 IgG2a clone 29F.1A12, the deglycosylated rat anti-mouse PD-1 IgG2a, and nivolumab (Bristol-Myers Squibb) with a covalently-attached and photo-stable Alexa Fluor 647. Dextran nanoparticles preferentially accumulate in macrophages and are commonly used as an in vivo macrophage imaging agent (23). Ferumoxytol (60 mg Fe, AMAG Pharmaceuticals) was dialyzed overnight against water, aminated overnight at r.t. in a 6.5 mL solution containing 1 g (N-3-dimethylaminopropyl)-N-ethylcarbodiimide hydrochloride and 0.6 nM ethylenediamine dihydrochloride, and dialyzed at pH 8 in a 0.02 M Na-citrate buffer containing 0.15 M NaCl. Pacific Blue-succinimidyl ester (5 mg, Thermo Fisher Scientific) was dissolved in DMF and 0.6 mL was incubated overnight with 2 mL aminated dextran nanoparticle at r.t. Free dye was removed using PD-10 columns (GE Healthcare). Pacific Blue-SE (Life Technologies) was also used to label 500 kDa amino-dextran (Thermo Fisher Scientific) according to the manufacturer's instructions. The 29F.1A12 rat anti-mouse PD-1, nivolumab, and human IVIG (a kind gift from Harry Meade, LFB USA) were natively deglycosylated in parallel using PNGase F (New England Biolabs) following manufacturers' protocols. The de-glycosylated antibodies were purified using Protein G beads (Pierce Biotechnology). Antibody glycan profiling was performed as previously described (46), using an Agilent HPLC 1260, with Agilent AdvanceBio Glycan Mapping HILIC-based column. Glycoforms were identified by referencing the elution times of the digested glycans from the IVIG standard. Rat anti-mouse CD16/32 clone 2.4G2 (BioXcell) was used for in vivo FcγR blocking (47).

Study Design

The objective of this study was to understand the binding tropism of aPD-1 mAb therapy in tumors. Flow cytometry, ex vivo microscopy, and intravital microscopy techniques were used for longitudinal investigation of checkpoint blockade pharmacokinetics. The expectation was that the fluorescently-tagged drug would allow tumor-infiltrating lymphocytes to be tracked during pharmacodynamic response, however accumulation of drug in tumor-associated macrophages prompted us to hypothesize that the Fc region of these monoclonal antibodies can also affect the drug biodistribution. All in vitro and in vivo studies were performed using C57BL/6J mice, and human primary cells were collected from healthy volunteers for ex vivo studies. Sample sizes were decided using data from preliminary caliper measurements of tumor growth (standard deviation=25% with a 99% difference between treatment and control groups, α=0.05, β=0.2). Data were analyzed by Grubbs' test for statistical outliers, which were pre-defined using an alpha value of 0.01. Data are representative of at least 3 independent experiments as indicated in the Figure legends. Treatment cohorts were assigned to ensure that tumors were size-matched at the start of the intervention. Caliper measurements were acquired by researchers blinded to the intervention and group allocation. Microscopy imaging was performed unblinded using predetermined data collection methods that allowed multiple regions to be studied for each sample, permitting thousands of cells to be analyzed.

Animal Models

Animal research was performed in accordance with the Institutional Animal Care and Use Committees at MGH. DPE-GFP mice (21) were generously provided by Ulrich von Andrian (Dept. of Microbiology and Immunobiology, Harvard Medical School), IFN-γ reporter with endogenous polyA tail (GREAT) mice (22) were kindly provided by Andrew Luster (Division of Rheumatology, Allergy and Immunology, Massachusetts General Hospital). Tumor growth was monitored by caliper measurement, and the area (A) of these predominantly two-dimensional tumors was calculated using the formula A=length*width. Tumor implantation was performed by intradermal injection of tumor cells (2×10⁶ MC38/MC38-H2B-mApple, 5×10⁵ B16-F1-Ova, and 5×10⁵ KP1.9). MC38 cells were gifted by Dr. Mark Smyth (QIMR Berghofer, Brisbane, Australia), B16 cells were from ATCC, and KP1.9 cells were a gift from Dr. Alfred Zippelius (University Hospital Basel, Switzerland). Experiments were generally started when tumors became vascularized, which was after 8 days. For aPD-1 and AF647-aPD-1 treatments, mice were given 200 μg i.p. of the 29F.1A12 aPD-1 clone. For in vivo Fc blocking experiments, mice were infused i.p. with 200 μg of monoclonal antibody specific to mouse Fc gamma receptors II and III (clone 2.4G2, Bioxcell) daily for 5 days. Control mice received 200 μg rat IgG2a isotype control (clone 2A3, Bioxcell).

In Vivo Microscopy

Intravital microscopy was performed in dorsal skin-fold window chambers installed on DPE-GFP or GREAT mice inoculated with MC38-H2B-mApple tumors. Mouse macrophages and/or vasculature were labeled with Pacific Blue ferumoxytol and dextran, respectively. AF647-aPD-1 (200m) was delivered i.v., and its tumor distribution was observed using an Olympus FluoView FV1000MPE confocal imaging system (Olympus America), as described previously (45). Pacific Blue, GFP/YFP, mApple, and AF647 were imaged sequentially using 405, 473, 559, and 635 nm lasers and BA430-455, BA490-540, BA575-620, BA575-675 emission filters with DM473, SDM560, and SDM 640 beam splitters, all sourced from Olympus America. Time lapse images were acquired continually over the first hour after AF647-aPD-1 injection, after which the mice were allowed to recover before subsequent imaging.

Cell Models

Cell lines were maintained in Iscove's growth medium supplemented with 10% heat inactivated Fetal Calf Serum (Atlanta Biologicals) and 100 IU penicillin, 100 m/ml streptomycin (Invitrogen) at 37° C. and 5% CO₂ and checked monthly for mycoplasma. The MC38 mouse colon adenocarcinoma cell lines were kindly provided by Mark Smyth (QIMR Berghofer Medical Research Institute). MC38 cells were transfected with an H2B-mApple reporter for imaging as previously described (48). Briefly, cells were cultured in a 24-well dish and transfected with the pLVX-H2B-mApple lentiviral vector (Clontech) in the presence of 10 μg/mL polybrene (Santa Cruz Biotech). Fresh medium was provided after 24 hours, and cells were split the following day into 3 μg/mL puromycin for selection. A population of transfected MC38-H2B-mApple was obtained by FACS sorting. The murine lymphoma EL4 cell line was acquired from ATCC. Suspended cells were passed before they reached densities exceeding 1×10⁶ cells/mL. Murine bone marrow-derived macrophages (BMDMs) were isolated from surgically-resected femurs and tibias of C57BL/6J mice. Under sterile conditions, bone marrow was flushed from the bone using a PBS-filled syringe and a 28-gauge needle. Cells were centrifuged for 5 minutes at 300×g and red blood cells lysed with ammonium chloride at 4° C. for 5 minutes. The remaining cells were plated in growth medium supplemented with 10 ng/mL of murine macrophage-colony stimulating factor (M-CSF, Peprotech). M-CSF supplemented media was replaced every 2 days. Human peripheral blood mononuclear cells (PBMCs) from healthy donors were carefully isolated from buffy coats on a Ficoll-Paque PLUS gradient (GE Life Sciences) using the manufacturer's protocol. These cells were plated in petri plates and maintained in 50 ng/mL of human M-CSF (Peprotech) with media changes every 3 days. CD8⁺ lymphocytes were also isolated from donor-matched PBMCs using the Human CD8⁺ T Cell Isolation Kit (Miltenyi Biotec). CD8⁺ lymphocytes were cultured on 24-well plates that were pre-coated overnight with an anti-CD3 antibody clone OKT3 (BD Biosciences) to induce PD-1 expression. Cells were stimulated for 3 days before use in bioassays.

Fluorescence Reflectance Imaging

Gross examination of AF647-aPD-1 bio-distribution was performed in an Olympus OV100 imaging system. C57BL/6J mice were shaved and inoculated intradermally on the flank with 2×10⁶ MC38 tumor cells in 50 μL PBS. When tumors reached approximately 30 mm² (8 days), mice were assigned to treatment cohorts and given one injection of 200 μg AF647-aPD-1 intravenously. A vehicle control group received unlabeled rat IgG2a isotype control. Mice were sacrificed 30 minutes, 4 hours, 24 hours, or 72 hours post-treatment and tissues of interest were surgically resected, rinsed with saline, and imaged using an Olympus OV100 with brightfield acquisition (122 ms exposure time) or corresponding fluorescence filters (1000 ms exposure time). Mean fluorescence intensity values from ROIs manually drawn around each organ in ImageJ were background-corrected and reported as a ratio relative to the control-treated cohort.

Flow Cytometry

Spleen and lymph nodes were minced and passed through 40 micron filters (BD Falcon), whereas lung and tumor tissue were first digested in RPMI containing 0.2 mg/ml collagenase II (Worthington) at 37° C. for 30 minutes and then passed through a 40 micron filter. Red blood cells were lysed using ACK lysis buffer (Thermo Fisher Scientific). Cells were pre-treated with FcγR block (TruStain FcX anti CD16/32 clone 93, BioLegend) before staining with fluorochrome labeled antibodies, CD90.2 (53-2.1, BD), CD11b (M1/70, Biolegend), CD8a (53-6.7, BD), CD45 (30-F11, BD), F4/80 (BM8, Biolegend), CD11 c (N418, Biolegend), CD4 (RM4-5, BD), NK1.1 (PK136, BD), B220 (RA3-6B2, BD), in buffer containing 0.5% BSA and 2 mM EDTA. 7AAD was used to exclude dead cells from analysis. EL4 cells were used as a positive control for aPD-1 staining in our analyses. For antibody transfer/internalization studies, surface aPD-1 was removed using an acid wash (RPMI 1640, 2% FCS, pH 2) method previously described (49). Re-probing the T cells with PE-aPD-1 (RMP1-14, Biolegend) was used to quantitate remaining surface expression of PD-1 protein. Rat anti-mouse PD-1 was also detected in tumor tissue using anti-rat IgG2a (R2A-21B2, eBiosciences) with the following fluorochrome-labeled rat anti-mouse IgG2b antibodies: CD8b (YTS156.7.8, Biolegend), MHCII (M5/114.15.2, eBioscience), CD45 (30-F11, eBioscience), CD11b (M1/70, BD), and Hamster anti-mouse CD11 c (N418, Biolegend). To block FcγRs, these cells were pre-treated with rat IgG2b anti-mouse CD16/CD32 (2.4G2, Tonbo) and purified mouse IgG1 anti-mouse CD64 (X54-5/7.1, Biolegend). Cells were fixed and permeabilized using CytoFix CytoPerm (BD) for intracellular staining. Samples were run on a LSR II flow cytometer (BD) and analyzed using FlowJo software (Treestar).

Intravital Imaging

Mice were anesthetized and hair on the flank removed by shaving and 30 seconds of NAIR application. Dorsal skin-fold window chambers were installed as previously described (45, 50) and mice were kept on analgesic for the next 72 hours. One day after window placement, the top skin layer was removed using sterile instruments. At least 24 hours was allowed for resolution of swelling in the window chamber, then MC38-H2B-mApple cells (2×10⁶ in 20 μL) were injected in the fascia layer. Tumor growth was carefully monitored for the next 7 days. Time-lapse imaging was performed 8 days after inoculation. Pacific Blue-dextran nanoparticle (containing 1 nmol Pacific Blue dye) was injected i.v. 24 hours prior to imaging for macrophage labeling. A 30-gauge catheter was placed in the tail vein of an anesthetized mouse (2% isoflurane in oxygen) and Pacific Blue-dextran (containing 37 μg dextran and 56 nmol Pacific Blue dye) was injected for vascular labeling. Mice were maintained under anesthesia on a heating pad kept at 37° C. and imaged using an Olympus FluoView FV1000MPE confocal imaging system (Olympus America). Vascularized regions were selected using a 2× air objective XL Fluor 2×/340 (NA 0.14; Olympus America), before switching to a XLUMPLFL 20× water immersion objective (NA 0.95; Olympus America) for high resolution imaging with 2× digital zoom. Z-stack acquisition settings (e.g. voltage and laser power) were optimized for sequential scanning of 5 μm slices using 405, 473, 559, and 635 nm lasers paired with a DM405/473/559/635 nm dichroic beam splitter. The beam splitters (SDM473, SDM560, and SDM 640) and emission filters (BA430-455, BA490-540, BA575-620, BA575-675) required for acquisition of emitted light were sourced from Olympus America. For pharmacokinetic analysis, a time-course using 8 ms scan speed for a total frame interval of 183 seconds was acquired at two non-overlapping coordinates, during which the AF647-aPD-1 mAb was delivered via catheter. Cell motility measurements were made 20 minutes before and 20 minutes after AF647-aPD-1 injection using time lapse images acquired with higher temporal resolution (frame interval of 21 seconds). The Manual Tracking Plugin included in the FIJI image processing package was used to characterize cell tracks. Cell motility coefficients (M) were calculated from each cell track using the slope of the regression function fitted to the mean displacement plot, according to the following formula: M=d²/4t, where d is displacement from origin at time t (51). Track plots were creating using the Chemotaxis and Migration Tool for ImageJ (IBIDI).

Live Cell Imaging

BMDMs cultured for 6 days in 35 mm poly-D-lysine coated 14 mm glass-bottom 35 mm dishes (Mattek, Ashland, Mass.) were stained with the PKH26-Red staining kit (Sigma-Aldrich) following the manufacturer's protocol. PD-1 expressing EL-4 T cells were stained with the PKH67-Green Fluorescent Cell Linker kit (Sigma-Aldrich), then stained with either AF647-rat IgG2a isotype control (InVivoMAb), AF647-aPD-1 mAb, or de-glycosylated AF647-aPD-1 mAb as indicated. After thorough washing, labeled T cells were added in co-culture to the BMDM dishes placed within the 8 carousel positions of a VivaView FL incubator fluorescence inverted microscope (LCV110, Olympus America). Images were acquired in each sample every 5 minutes using a 20× objective with 0.5× zoom, and live cell tracking was performed in the presence of vehicle control, Fc block (TruStain FcX), anti-FcγRIV 9e9 antibody (generously provided by Robert Anthony) (52), or Dynasore (80 μM, Sigma-Aldrich). Human PBMC/CD8⁺ T-cell co-cultures were stained using the same PKH26-Red and PKH67-Green kit protocols described for murine BMDM:T cell cultures, and AF647-nivolumab was used to stain anti-CD3 stimulated CD8⁺ lymphocytes. Human Fc receptor block (TruStain FcX, BioLegend) was used where indicated.

Image Processing

Image files were prepared for Figure panels using the FIJI package of ImageJ for pseudo-coloring fluorescent channels, adjusting background/contrast, and creating Z-projections. For in vivo object segmentation, rolling ball background subtraction and thresholding using the Otsu method were used to create object masks for cells and vessels. AF647-aPD-1 intensity levels were measured within ROIs created from the indicated object masks. At low concentrations, fluorescence intensity approaches linearity with concentration, and the fluorescence intensity ratio at time (Iₜ) and pre-injection (I₀) was used to report Cₜ/C₀. In vitro image analysis, including PD-1⁺ puncta quantification, was performed using a custom CellProfiler pipeline. Macrophages were segmented using the Otsu threshold method and macrophages that did not come in contact with lymphocytes were excluded. The speckle counting function in CellProfiler was used to quantitate puncta.

Rat IgG2a Detection Assay

EL-4 T cells were labeled with AF647-aPD-1 and co-cultured for 2 hours with mouse bone marrow derived macrophages (BMDM). The supernatant containing non-adherent T cells was removed and the BMDM monolayer was washed 3× with PBS. Flow cytometric analysis was used to confirm removal of T cells. Fresh media was then added to the BMDM monolayers and cells were incubated for 0 or 4 hours before being scraped, collected into microcentrifuge tubes, and frozen overnight. Samples were concentrated using Amicon Ultra-0.5 mL Centrifugal Filters with a 30 kDa cut-off (Millipore), and the rat IgG2a content measured using the Rat IgG ELISA kit (eBioscience) following the manufacturers instructions and a Tecan Safire 2 fluorescence plate reader. Co-cultured cells for flow cytometry were scraped in MACS buffer, stained with F4/80 and CD90 antibodies to distinguish BMDM from T cells, and the AF647 binding tropism was independently measured at the indicated time points.

Statistical Analysis

Data points were compiled in Microsoft Excel, and statistical analyses were performed using GraphPad Prism 6. Alpha levels of 0.05 were used to define statistical significance, and error bars represent SEM unless otherwise noted.

### Example 1. Global aPD-1 mAb Biodistribution

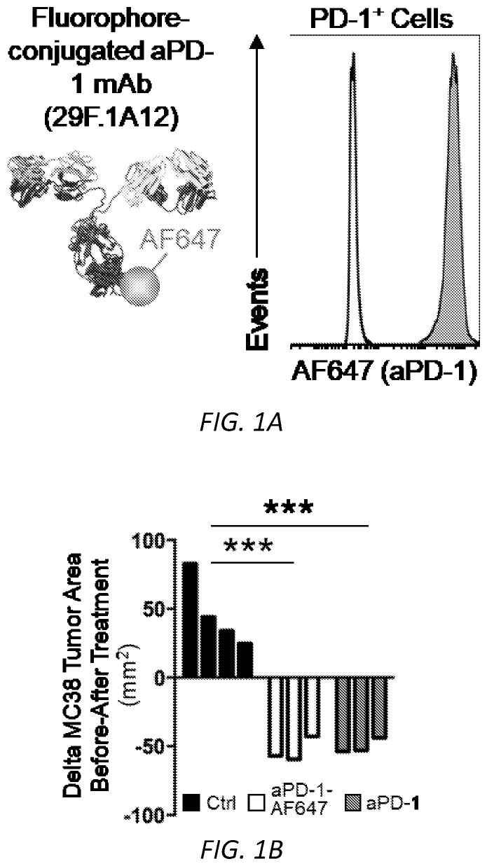

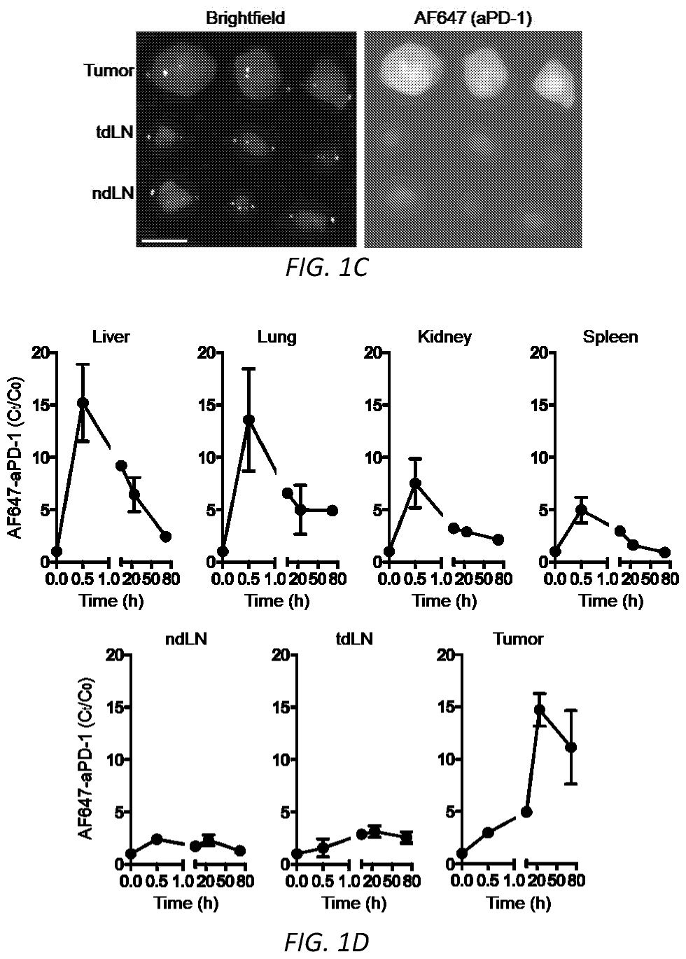

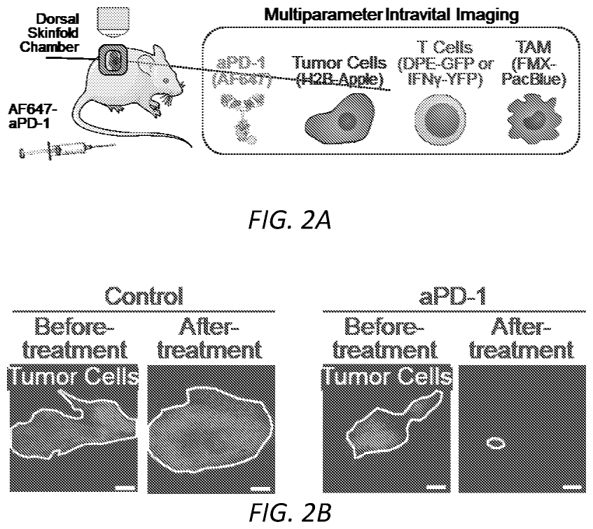

We initially sought to track the temporal distribution of aPD-1 mAbs in vivo at the organ level. We thus covalently labeled aPD-1 mAb (clone 29F.1A12) with an Alexa-Fluor 647 dye (AF647-aPD-1) using N-Hydroxysuccinimide (NETS) chemistry. For maximal brightness without dye quenching, we optimized the labeling conditions to achieve ˜4 fluorochrome molecules per antibody. For in vitro studies, the EL-4 mouse lymphoma cell line was used as a T cell model because of its stable PD-1 expression and its broad adaptation to in vitro culture (19). Using this cell line, we confirmed that fluorescent labeling of aPD-1 did not interfere with the drug's binding specificity (FIG. 1A). Additionally, AF647-aPD-1 retained therapeutic activity in the ovalbumin-expressing MC38 tumor model, which is responsive to single-agent aPD-1 therapy (FIG. 1B) (20). Collectively, these data indicate that AF647-aPD-1 retains PD-1 tropism and anti-tumor activities.

We next examined the in vivo biodistribution of AF647-aPD-1 in the wild-type MC38 tumor model, which also responds to aPD-1 treatment, but less efficiently than the ovalbumin-expressing MC38 counterpart in which aPD-1 treatment results in uniform tumor rejection. Mouse cohorts were sacrificed at times ranging from 0.5 to 72 hours after treatment, and organs were removed for fluorescence measurements (FIG. 1C). AF647-aPD-1 signal was primarily retained within tumors over time (FIG. 1D). We observed a large spike of AF647-aPD-1 in the liver, lungs, kidney, and spleen at 0.5 hours post-injection, followed by a subsequent decrease over time that coincided with increases in AF647-aPD-1 signal in the tumor (FIG. 1D). Collectively, these data indicate that the drug started to accumulate in tumors within minutes after injection but that maximal aPD-1 accumulation in the tumor was achieved after 24 hours.

### Example 2. Cellular Kinetics and Dynamics of aPD-1

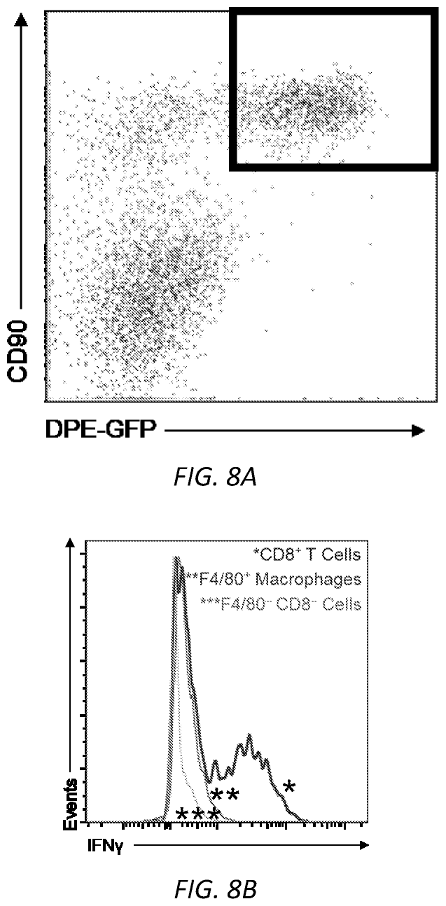

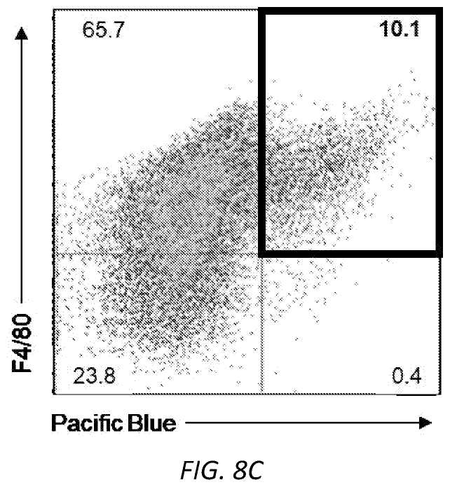

After observing that aPD-1 mAbs collect within the tumor microenvironment shortly after administration, we further aimed to study whether the drugs bind their intended target T cells at the tumor site. To this end, we used intravital microscopy in dorsal skin-fold chambers, which enabled us to examine the distribution and tropism of AF647-aPD-1 at subcellular resolution within the tumor stroma and longitudinally after drug administration (FIG. 2A). The experimental system allowed simultaneous tracking of four components: aPD-1 mAbs (labeled with AF647); MC38 tumor cells (labeled with H2B-mApple); T cells (labeled with GFP or YFP); and tumor-associated macrophages (labeled with PacificBlue-dextran nanoparticles). We used two different reporter mouse models to visualize T cells: i) DPE-GFP mice (21) in which all GFP-expressing cells are CD90⁺ (FIG. 8A), and ii) interferon gamma reporter (GREAT) mice (22), which were useful because tumor-infiltrating YFP+ cells in these mice were almost exclusively CD8⁺ T cells (FIG. 8B). The fluorescent PacificBlue-dextran nanoparticle has been validated for intratumoral macrophage identification (23) and we confirmed its specificity for macrophages (F4/80⁺ cells) in the tumor stroma (FIG. 8C). Finally, we verified that single agent aPD-1 treatment was able to suppress MC38-H2B-mApple tumor growth in the window chamber system (FIG. 2B).

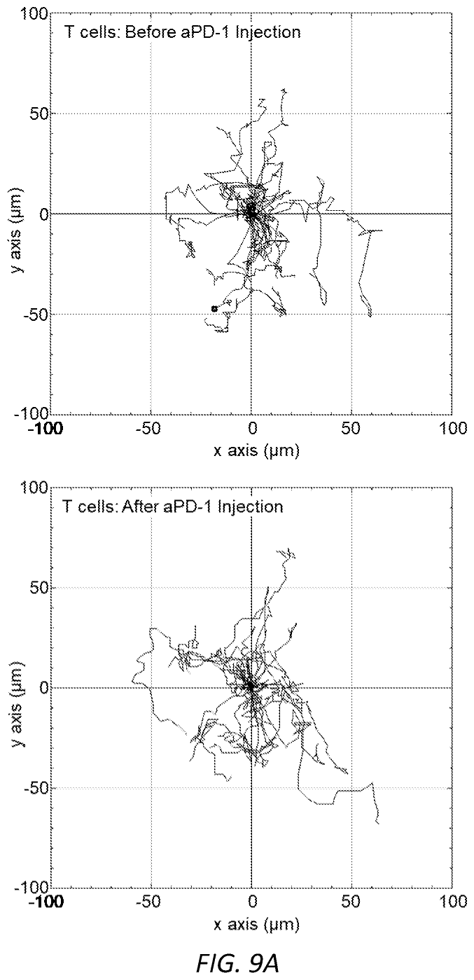

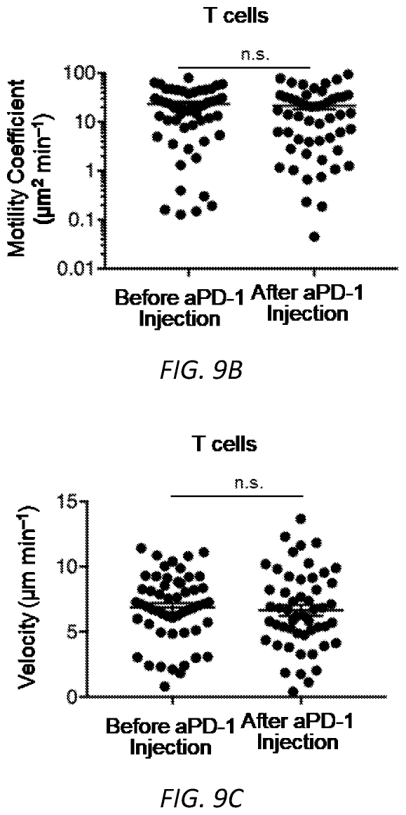

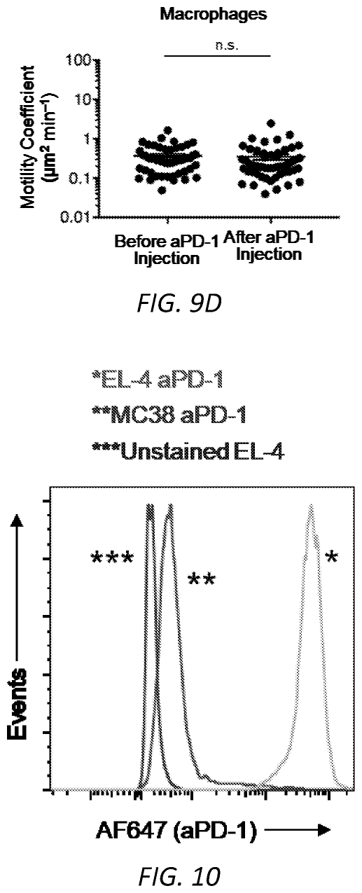

Upon administration of AF647-aPD-1, we found that the antibody rapidly perfused tumor vessels and gradually disseminated out of the vasculature and into the tumor interstitium. AF647-aPD-1 was observed on GFP-labeled T cells as early as 5 minutes after injection, and these were the first cells in the tumor microenvironment to be detectably labeled by the drug. AF647-aPD-1 binding to tumor-infiltrating T cells was initially peri-cellular, but within minutes formed puncta on the cell surface. These rearrangements occurred without apparent decreased T cell motility (FIGS. 9A-9C). Later time points revealed that tumor-associated macrophages, which were stationary in the tumor microenvironment (FIG. 9D), had collected most of the AF647-aPD-1; T cells were not associated with AF647-aPD-1 at these time points (FIG. 2C). T cells were present in the tumor microenvironment at all times examined, removing the possibility that the drug biodistribution was an artifact of T cell loss after therapy. Also, tracing AF647-aPD-1 across all time-points failed to show binding to tumor cells, precluding the possibility that aPD-1 mAbs had direct effects on cancer cells (FIG. 10).

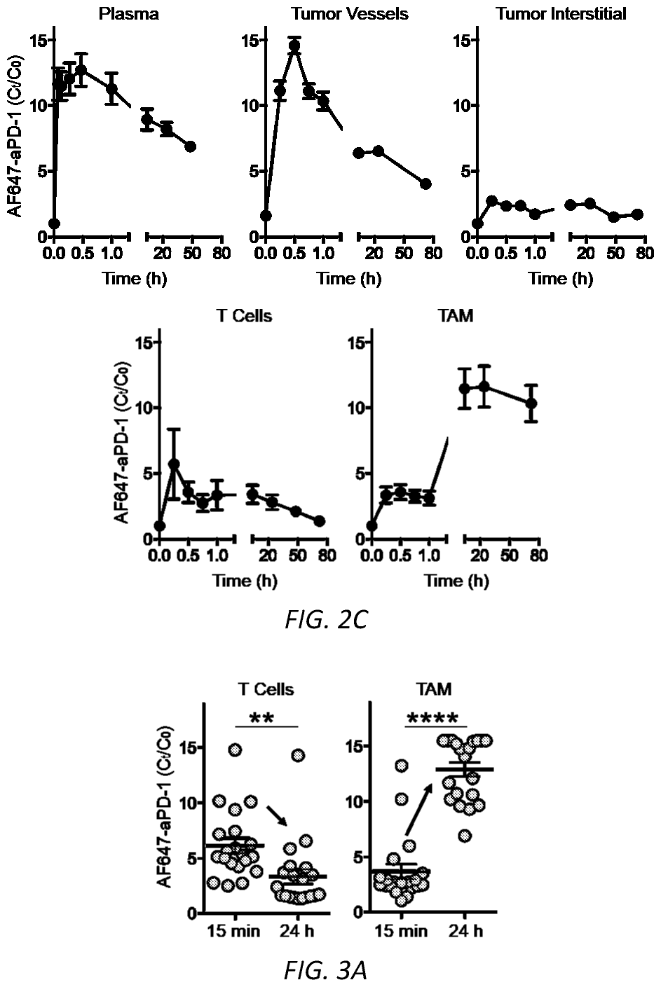

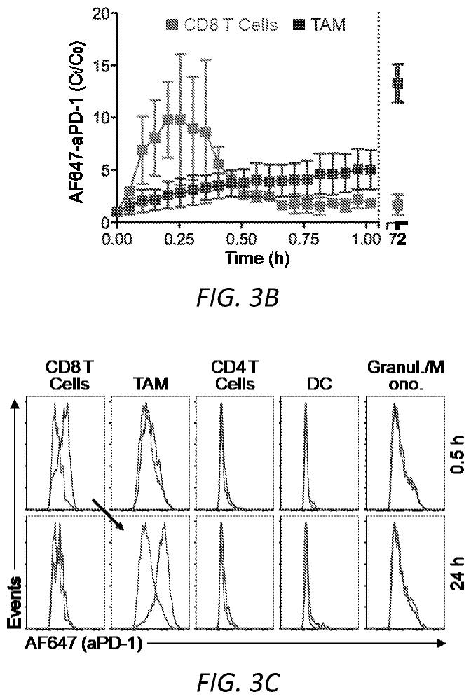

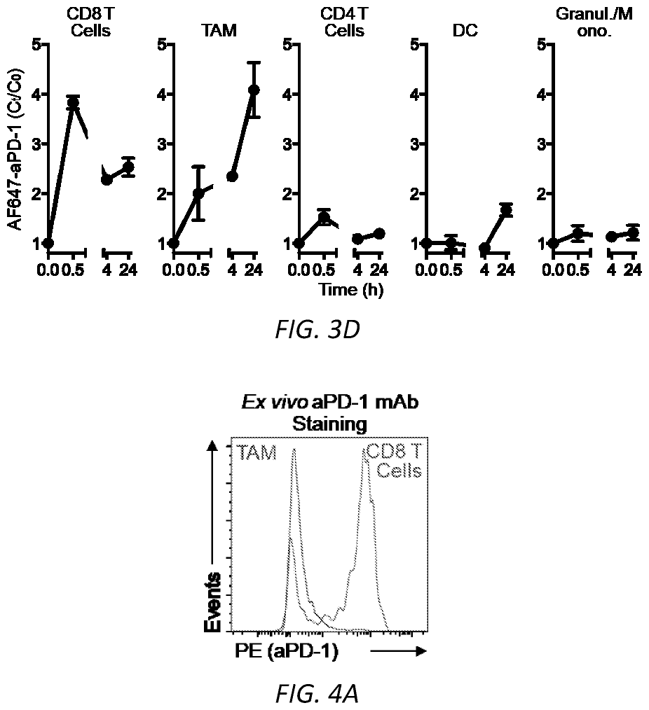

Quantification of the tumor microenvironment images taken at 15 minutes or 24 hours after drug administration showed a pattern of AF647 signal loss on T cells over time, and a concomitant AF647 signal increase on macrophages (FIG. 3A). While DPE-GFP labels CD90⁺ lymphocytes (FIG. 8A), the GREAT mouse model allowed us to focus specifically on IFNγ-expressing cells, which are almost exclusively CD8⁺ T cells in the tumor microenvironment (FIG. 8B). Intravital imaging of these cells confirmed surface binding of AF647-aPD-1 within minutes after drug administration. Longitudinal studies further showed that T cells eventually lose AF647-aPD-1 mAbs, which are physically transferred to, and retained by, neighboring macrophages. Consistent with our observations in DPE-GFP mice, we found in IFNγ reporter mice that aPD-1 transfer from T cells to macrophages limited the overall duration of drug binding to their intended target cells (FIG. 3B).

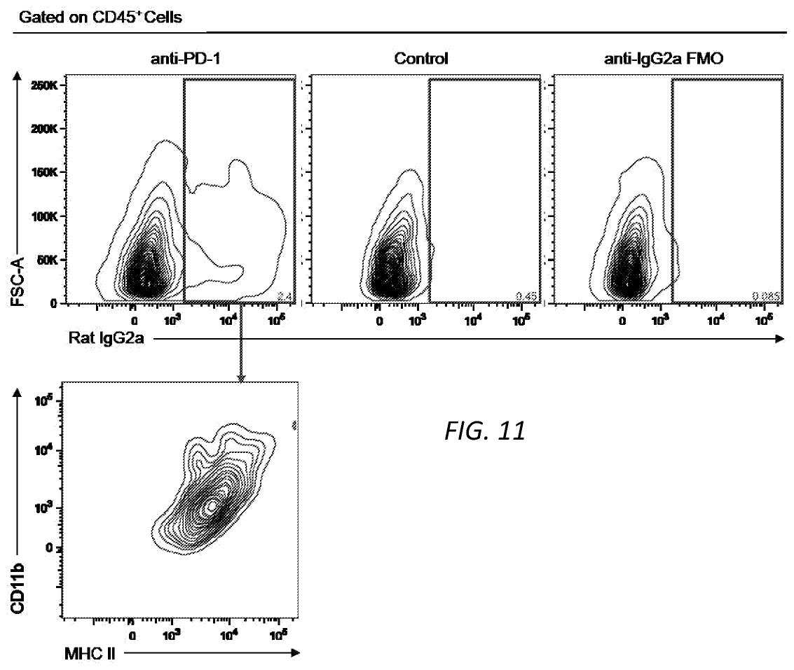

To address whether checkpoint blockade agent uptake by macrophages could be independently validated using a bulk tissue measurement, we performed flow cytometry of tissues excised from tumor-bearing animals at 0.5 and 24 hours after AF647-aPD-1 administration (FIG. 3C). We evaluated several relevant cell populations for aPD-1 binding and confirmed our intravital microscopy observations: aPD-1 mAbs were bound mostly to CD8⁺ T cells at 0.5 h, but to macrophages at 24 hours (FIG. 3C-3D). Tumor macrophages were positive for rat IgG2a (FIG. 11), confirming that the aPD-1 mAb, and not just the fluorophore, was transferred in vivo. Other cell types investigated, including CD4⁺ T cells, CD45⁺CD11b⁺F4/80⁻ cells (which can include granulocytes and monocytes), and dendritic cells did not display significant binding of aPD-1 at any time-point tested (FIGS. 3C-3D).

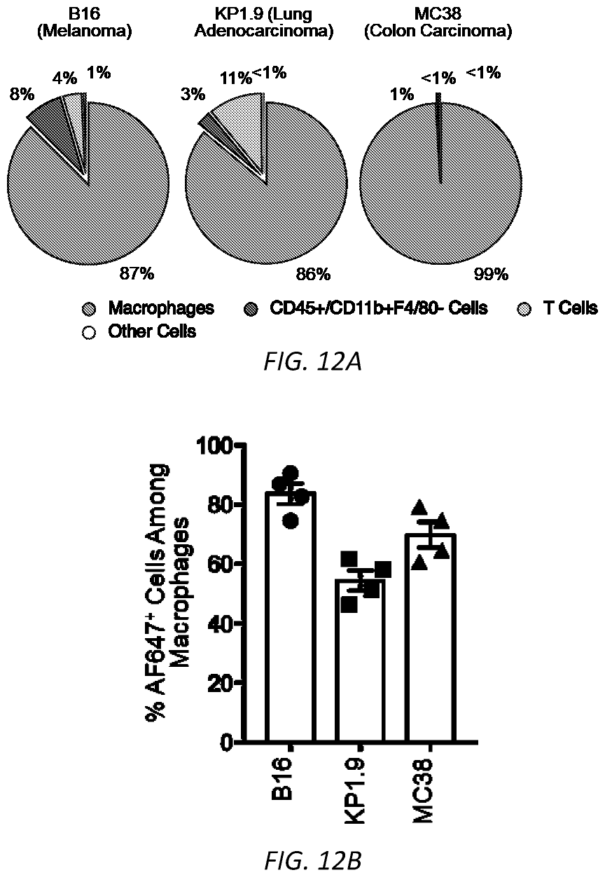

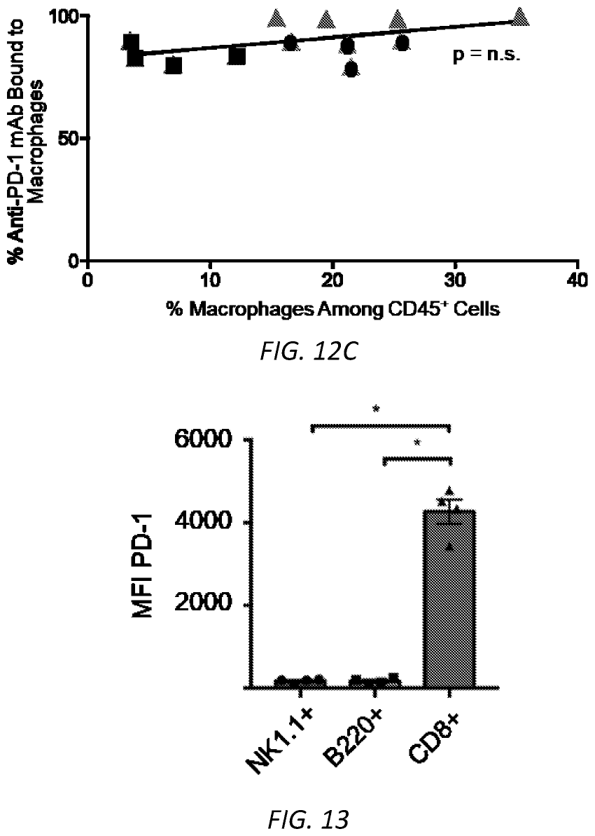

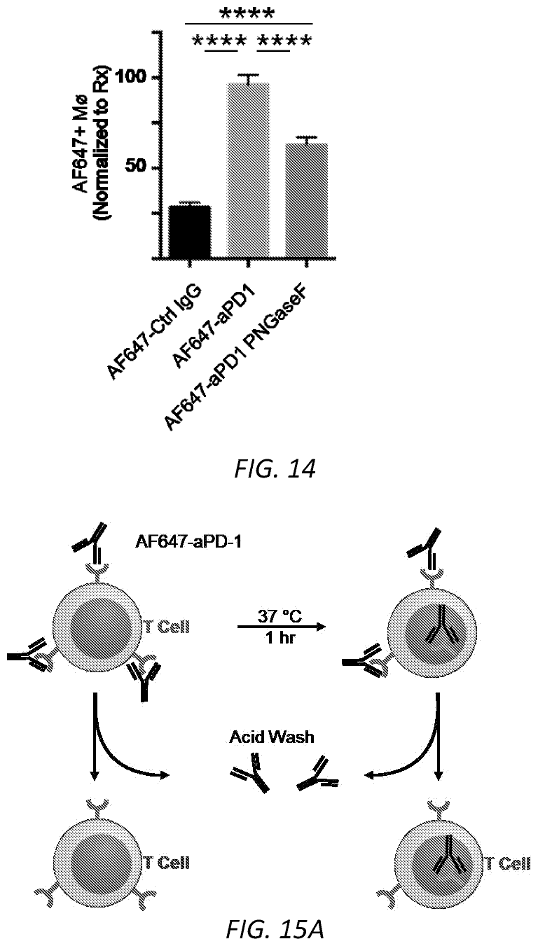

Analysis of B16 melanoma and KP1.9 lung adenocarcinoma tumor models also demonstrated that most AF647-aPD-1 mAbs accumulate within tumor-associated macrophages at 24 hours, similar to findings obtained with MC38 colon adenocarcinoma (FIG. 12A). The majority of intratumoral macrophages were bound by AF647-aPD-1 in all three tumor models (FIG. 12B). Linear regression analysis of combined data further indicated that anti-PD-1 mAb uptake by macrophages is independent of macrophage number and can also occur when tumor-associated macrophage numbers are relatively low (FIG. 12C).

### Example 3. aPD-1 mAb Transfer Mechanism

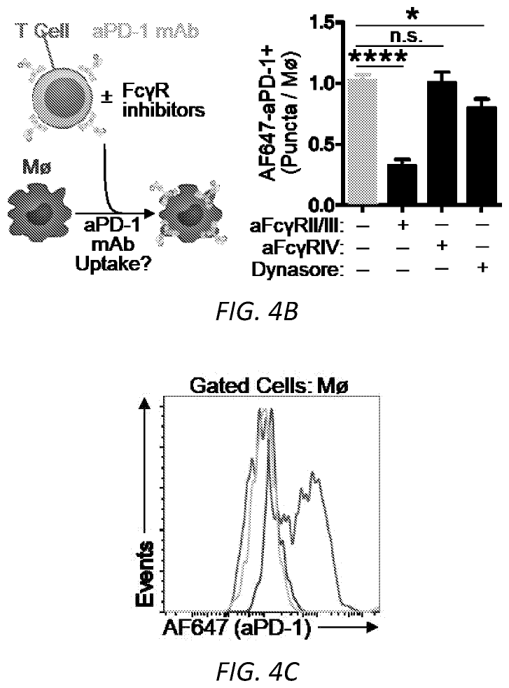

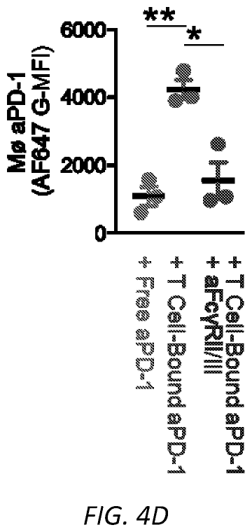

To explore the removal of aPD-1 mAbs on T cells by macrophages, we first asked whether the latter may also express PD-1 on their surface. However, ex vivo flow cytometry analysis indicated that tumor-associated macrophages, in contrast to tumor-infiltrating CD8⁺ T cells, were PD-1⁻ (FIG. 4A). PD-1 was also absent in NK and B cells in these tumors (FIG. 13). We then designed an in vitro co-culture system combining bone marrow derived macrophages and T cells that constitutively express PD-1 to create a controlled system to study the mechanism of drug collection by macrophages (FIG. 4B). T cells were pre-incubated with AF647-aPD-1 to emulate the antibody initially bound to T cells we observed in vivo, and then co-cultured with macrophages. Using this experimental setting, AF647-aPD-1 mAbs effectively relocated from T cells to macrophages within several minutes, as detected by the formation of drug puncta within macrophages (FIG. 4B). The transfer could not be attributed solely to phagocytosis of cell debris because it occurred even in the presence of the phagocytosis inhibitor dynasore (FIG. 4B). No macrophage uptake was observed when using an AF647-labeled isotype control IgG antibody, which did not bind T cells.

Since tumor macrophages did not capture AF647-aPD-1 in substantial quantities early after administration, did not express PD-1 on their cell surface, and withdrew drug bound to the T cell surface, we reasoned that AF647-aPD-1 mAbs must accumulate in macrophages through a non-antigen specific mechanism. The aPD-1 clone 29F.1A12 is a rat IgG2a isotype that is used to mimic the biological properties of human IgG4. Both rat IgG2a and human IgG4 have been demonstrated to bind inhibitory FcγRs (mouse FcγRIIb and human FcγRIIB, respectively) (24, 25). Adding FcγRIIb/III blocking antibodies to the in vitro co-culture system diminished AF647-aPD-1 transfer from T cells to macrophages (FIG. 4B). The effect was specific because blocking FcγRIV, an Fc receptor that does not bind rat IgG2a, failed to inhibit AF647-aPD-1 transfer (FIG. 4B).

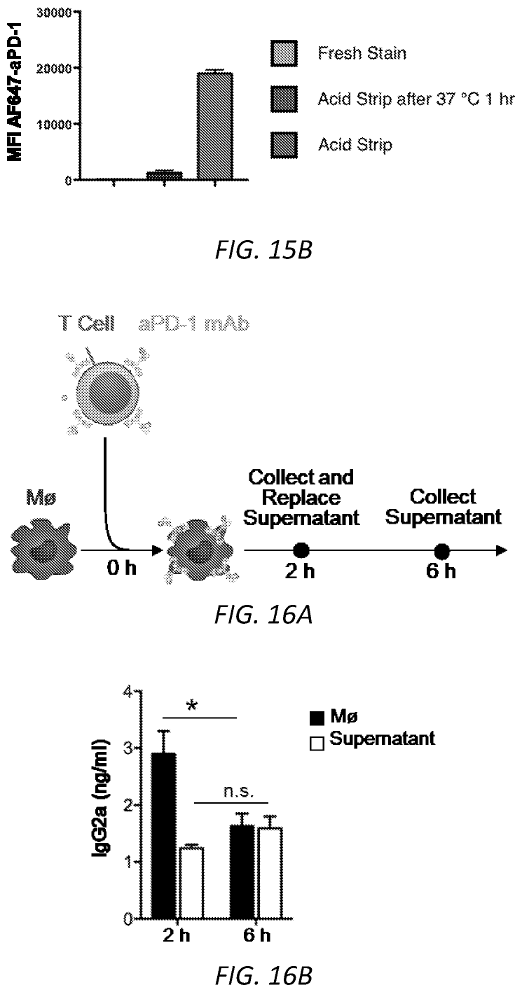

To substantiate these findings, we developed a flow cytometry-based antibody transfer assay: macrophages were incubated with T cells previously labeled with AF647-aPD-1 mAbs for 30 min and analyzed by flow cytometry. AF647-aPD-1 signal was detected on macrophages in this experimental setting (FIG. 4C); however, aPD-1 transfer could be neutralized by adding a blocking antibody to FcγRIIb/III to the co-culture system (FIG. 4C-4D). AF647-aPD-1 mAbs added directly to the culture medium in the absence of T cells was not efficiently taken up by macrophages, further suggesting that T cells are the major source of aPD-1 for macrophages (FIG. 4C-4D). We also tested whether aPD-1 loss on the T cell surface might be due to receptor internalization independent of macrophage uptake. T cells were exposed to AF647-aPD-1 for 1 h at 37° C., treated with an acid solution to remove cell surface antibodies, and analyzed by flow cytometry to assess remaining (internalized) fluorescent signal (FIG. 15A) (26). Acid stripping strongly reduced AF647-aPD-1 detection, indicating that antibody internalization is likely not the primary contributor to aPD-1 loss on T cells (FIG. 15B).

Macrophages co-cultured with AF647-aPD-1-coated T cells were positive not only for AF647, but also for rat IgG2a, as assessed by ELISA (FIG. 16A). The eventual decline in macrophage rat IgG2a was not accompanied by release of IgG2a into the supernatant, suggesting that acquired antibody is eventually degraded by the macrophage (FIG. 16B). Collectively, these data indicate that aPD-1 mAb removal from the T cell surface receptor by macrophages is a pharmacologic end-point elicited by FcγR interactions with T cell-bound antibody complexes.

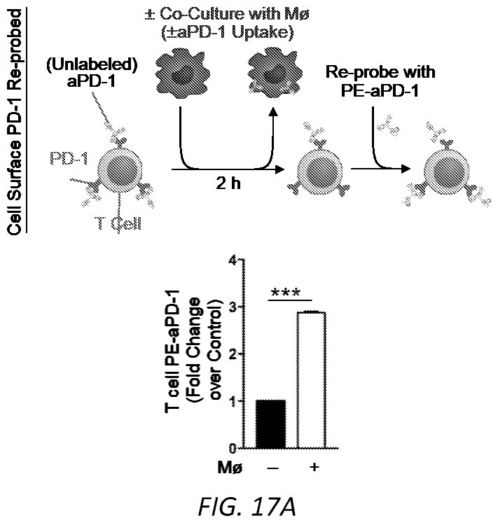

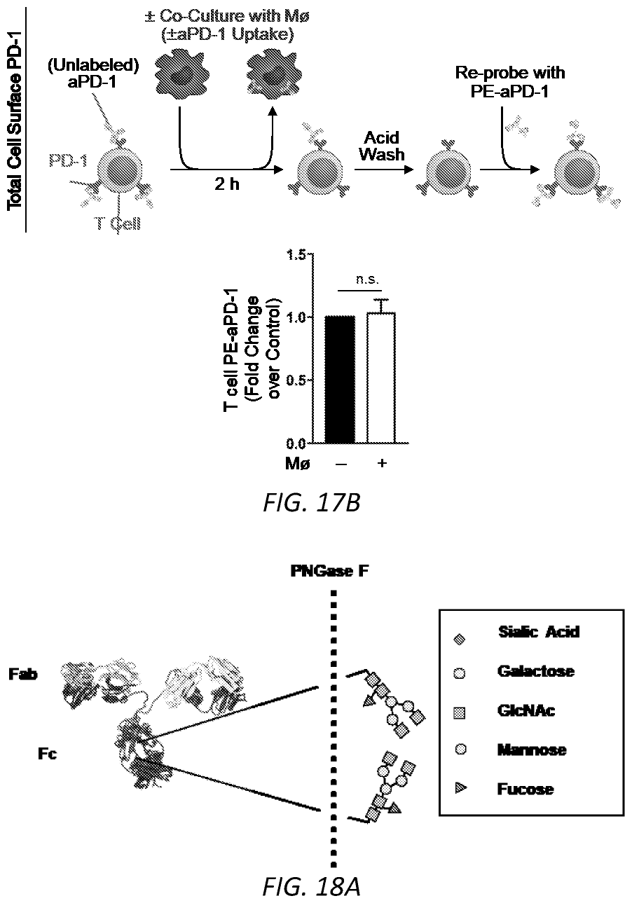

To assess whether the PD-1 receptor is transferred during aPD-1 removal, PD-1⁺ T cells were exposed to unlabeled aPD-1, co-cultured with macrophages (to enable aPD-1 capture), and re-probed for cell surface PD-1 expression with a fluorescent aPD-1 mAb. Transfer of unlabeled aPD-1 together with PD-1 would prevent PD-1 detection upon re-probing with fluorescent aPD-1. Instead, T cells from which aPD-1 had been captured were efficiently re-probed, indicating that aPD-1 removal frees up PD-1 molecules, which become available to fluorescent aPD-1 mAb (FIG. 17A). Control experiments confirmed that the increased re-probing signal was not contributed by new PD-1 molecules on the surface of T cells (FIG. 17B). Taken together, these data indicate that PD-1 remains on the T cell membrane after aPD-1 capture.

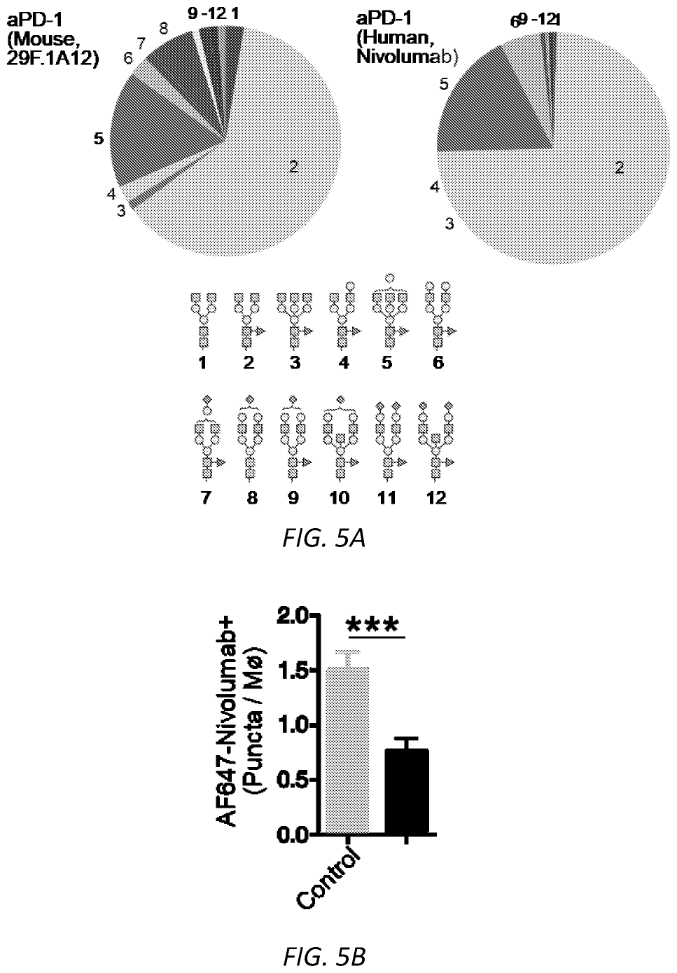

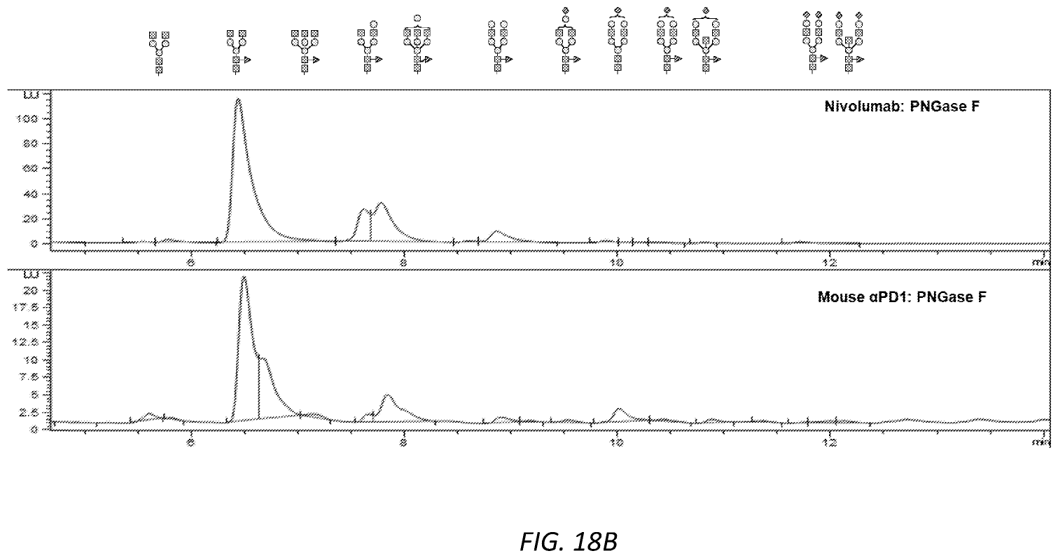

Because Fc:FcγR binding interactions of many IgG subclasses depend upon mAb Fc glycosylation (27), we profiled the glycan structures from the murine aPD-1 mAb (29F.1A12) and further extended our analysis to the human aPD-1 mAb nivolumab. Both antibodies were treated with PNGase F, a glycosidase that cleaves N-linked glycan, to remove glycan from each antibody, and the digested products were analyzed by HPLC (FIG. 5A). We found that murine and human aPD-1 mAbs share the same predominant glycoform that lacks terminal galactose residues (GOF) and is fucosylated on the penultimate N-acetylglucosamine (G1cNac, FIG. 18A-18B). Both mouse and human aPD-1 contained substantial fractions of terminally galactosylated glycoforms, indicating a high degree of Fc glycan heterogeneity in these aPD-1 mAb preparations.

The similarity in glycan pattern between mouse and human aPD-1 mAbs led us to hypothesize that FcγR-mediated antibody transfer is relevant to human aPD-1 interactions. We fluorescently labeled the anti-human PD-1 mAb nivolumab with AF647, and adapted the in vitro co-culture system (FIG. 4B) to primary human cells. We differentiated human macrophages from blood monocytes using macrophage colony-stimulating factor (M-CSF), and PD-1-expressing CD8⁺ T cells were generated by isolating primary human CD8⁺ T cells and stimulating them with plate-bound aCD3 mAbs for 3 days. Co-culture of AF647-nivolumab-labeled human CD8⁺ T cells with macrophages resulted in mAb transfer from CD8⁺ T cells to macrophages, and the transfer was inhibited by blocking FcγRs using aFcγRIIB/III. There was no evidence of CD8⁺ T cell membrane components in the macrophages, consistent with the antibody alone being removed from the surface PD-1 receptor.

Furthermore, blocking FcγRs decreased AF647-nivolumab puncta inside of macrophages, implying that FcγRs also regulate nivolumab uptake in human cells (FIG. 5B).

### Example 4. Improving Immunotherapy

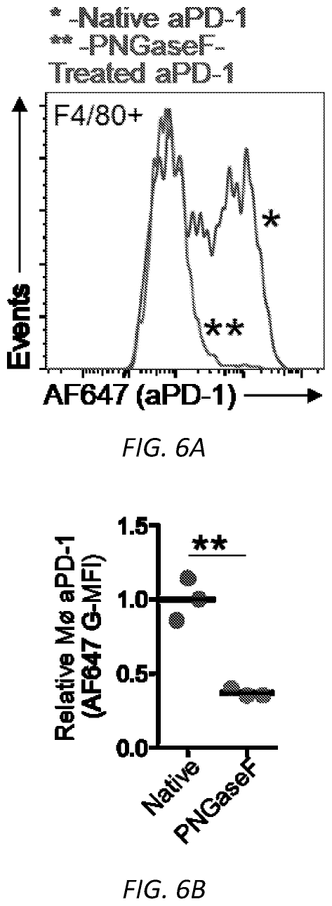

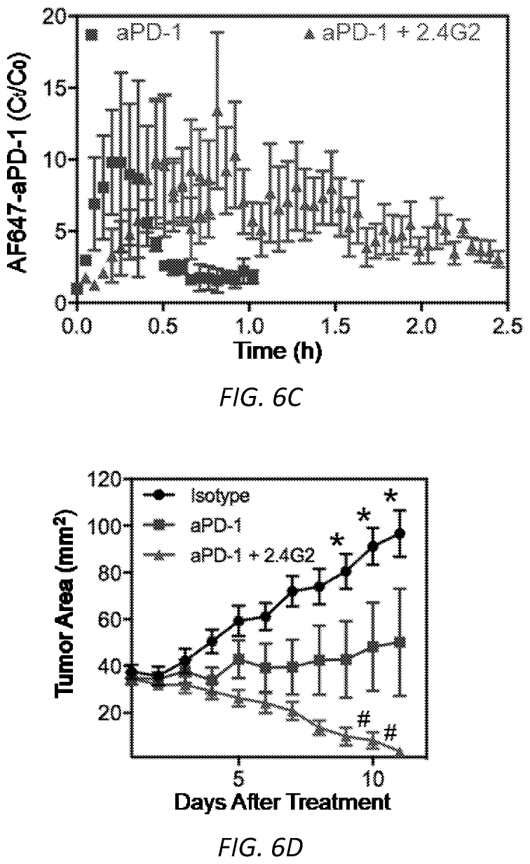

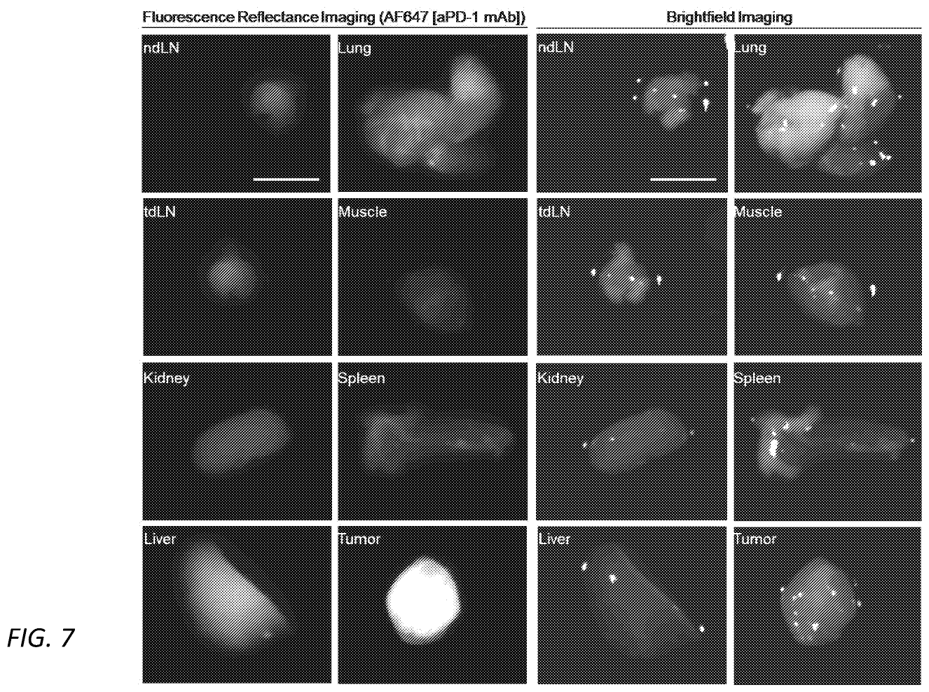

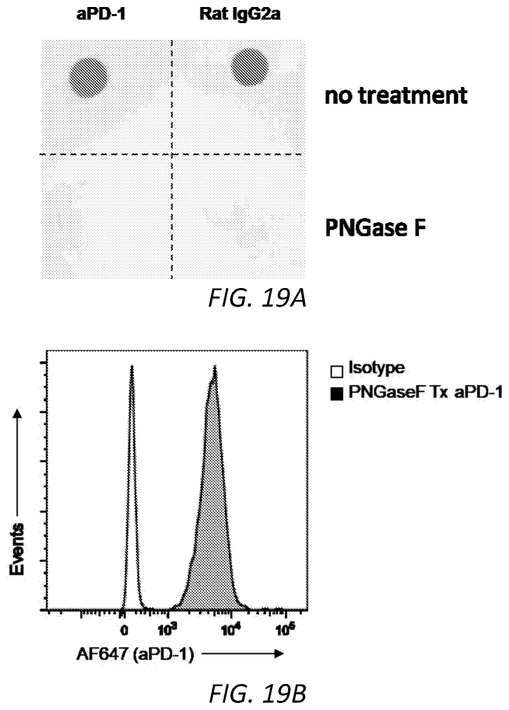

In an attempt to minimize aPD-1:FcγR interactions, we used PNGase F to remove the glycan from murine aPD-1 mAbs and confirmed cleavage of glycan by LCA lectin blot (FIG. 19A). The PNGase F-treated aPD-1 was labeled with AF647 and flow cytometry confirmed that the presence of glycan was not required for aPD-1 tropism as PD-1⁺ T cells were still efficiently labeled with PNGase F-treated AF647-aPD-1 (FIG. 19B). However, live cell imaging demonstrated that glycan removal diminished antibody transfer from T cells to macrophages, and these results were confirmed by flow cytometry (FIG. 6A-B).

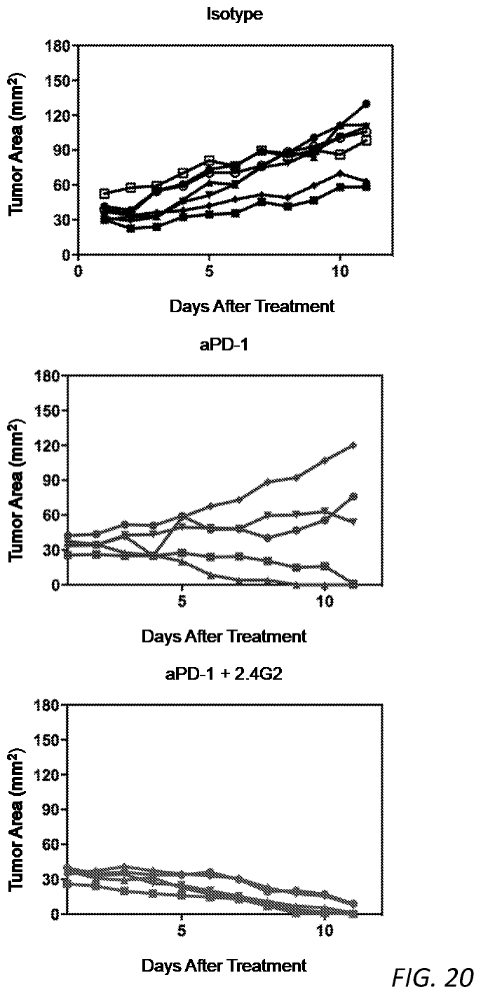

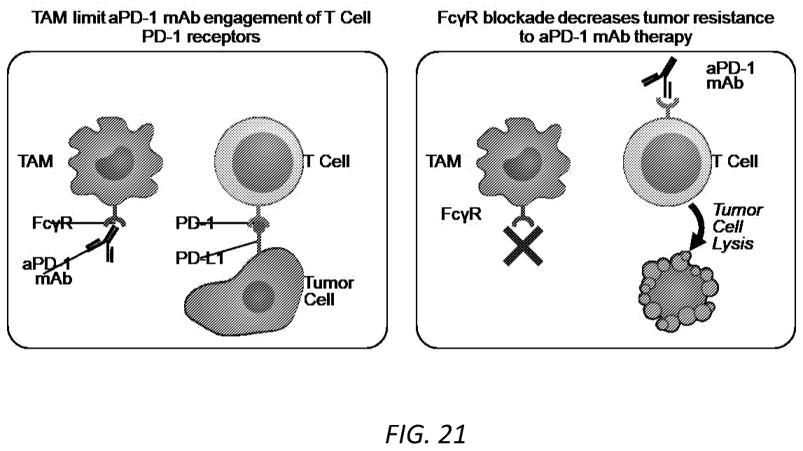

We then aimed to uncover the in vivo activity of aPD-1 mAbs when aPD-1: FcγR interactions were therapeutically inhibited. To this end, we tracked aPD-1, CD8⁺ T cells, macrophages, and tumor cells in mice in which we inhibited FcγRs by infusing FcγRIIb/III blocking Abs (2.4G2 clone) before delivering AF647-aPD-1 mAbs. We found that administration of the FcγR blocking agent substantially prolonged the occupancy time of AF647-aPD-1 mAbs on CD8⁺ T cells in the tumor bed (FIG. 6C). Furthermore, whereas the response of MC38 tumors to aPD-1 therapy typically varies among animals (FIG. 20), blocking FcγR interactions completely eliminated the fraction of non-responders observed, with complete tumor rejection in all mice that received the combination treatment (FIG. 6D). These data provide evidence that mAb:FcγR interactions abbreviate aPD-1 mAb occupancy time on tumor-infiltrating CD8⁺ T cells and limit therapy response; conversely, aPD-1 mAb therapy can be improved by blocking FcγR interactions (FIG. 21).

### Example 5. FcγRIIB Depleting/Inhibiting Strategies Alone do not Prevent Antibody Drug Removal by Macrophages

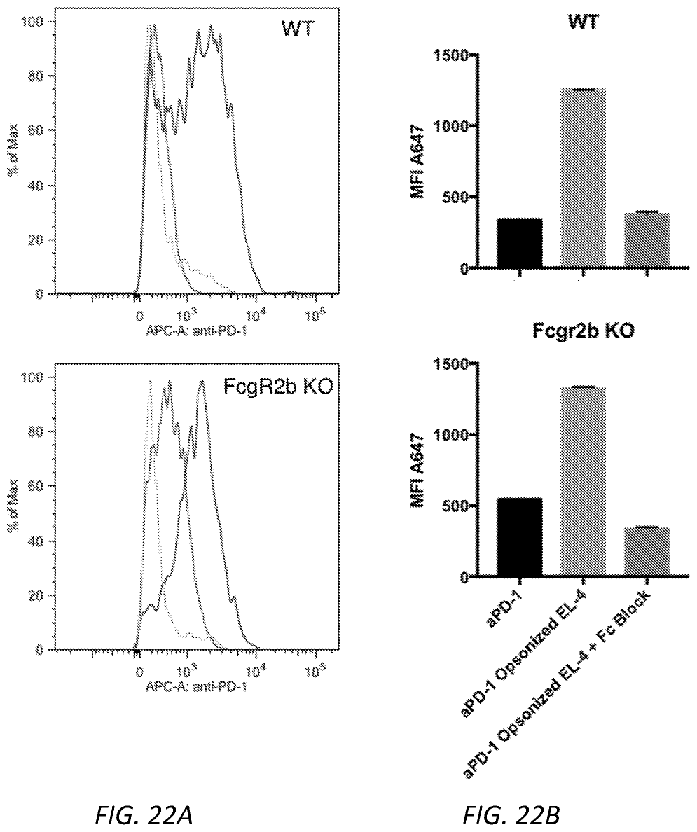

As shown in FIGS. 22A-22B, the mere absence of FcγRIIB did not prevent anti-programmed death receptor-1 (aPD-1) removal from T cell surfaces by macrophages in a macrophage, EL-4 co-culturing live imaging experiment. Despite a publication alleging that FcγRIIB−/− knockout mice respond better to aPD-1 therapy (Dahan et. al. Cancer Cell, 2015), the present data demonstrate that macrophages from FcγRIIB−/− mice exhibited no diminishment of aPD-1 transfer. Thus, the present methods are working by a different mechanism. Furthermore, in vitro imaging and flow cytometry data show that the resistance mechanism described here is not solely dependent on FcγRIIB, and FcγRIIB-neutralizing antibodies (Genentech) do not extend to the resistance mechanisms described herein.

### Example 6. Not all Antibodies of the Same Isotype are Subject to Macrophage Uptake

In experiments in mouse cells using a live imaging co-culture system with macrophages and EL-4 labeled with antibody, despite being the same antibody isotype as aPD-1(rat IgG2a), the anti-CD90 (clone 53-2.1) antibody was not subject to stripping from the T cell surface by macrophages. These data showed that rat antibody removal from murine macrophages is not a general artifact of the co-culture system. This is in contrast to the findings of Dahan et al. that D265a aPD-1 variants prevent mouse immune cells from recognizing rat IgGs.

### Example 7. Some Fc-Modifying Strategies Yield More Effective In Vivo aPD-1 Therapies

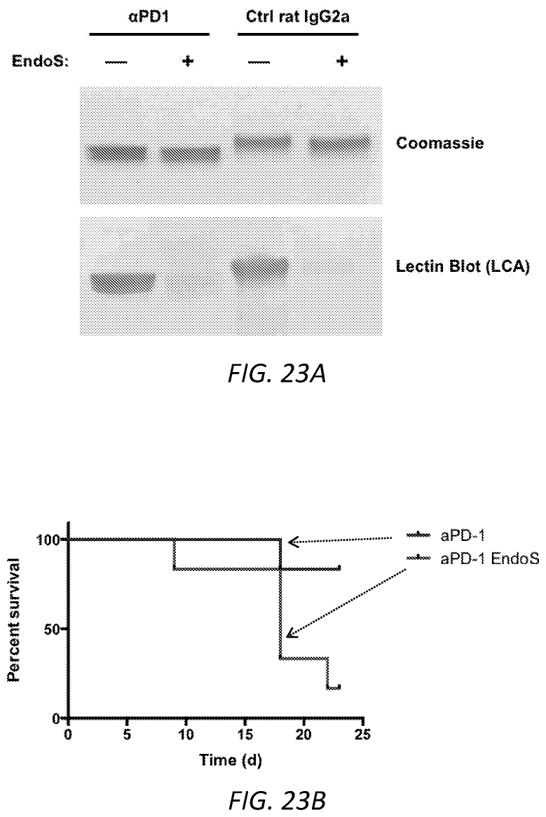

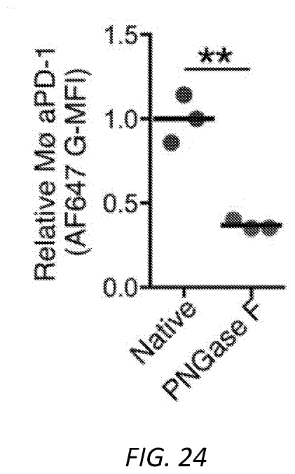

As shown in FIG. 23A, rat anti-mouse PD-1 (left) and isotype control (right) were treated with Endo S Agarose beads, for digestion of Fc associated glycan (lanes 2 and 4) and compared to native antibody (lanes 1 and 3). Coomassie staining following SDS-PAGE confirmed the shift in immunoglobulin size consistent with Endo S deglycosylation (top). Secondary confirmation of Endo S deglycosylated in lanes 2 and 4 was demonstrated using lens culinaris agglutinin (LCA) agent for lectin blotting (bottom). Surprisingly, as shown in FIG. 23B, our studies showed that deglycosylation using Endo S strategies did not improve survival in mice bearing MC38 colorectal tumors, which may indicate specificity of the methods described herein.

### Example 8. Reduction of Fcgamma Receptor Expression with Prednisolone

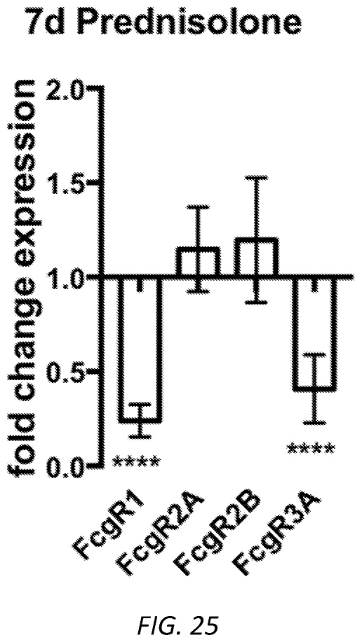

Prednisolone is an anti-inflammatory glucocorticoid. As shown in FIG. 25, flow cytometric analysis showed the expression of CD64 (FcγR1) and CD16 (FcγR3A) dramatically decreased during a 1 week time-course of prednisolone, the active form of prednisone. The low affinity CD32 Fc receptors (FcγR2A and FcγR2B) did not detectably change during this time-course. These data are representative of two healthy volunteers from which peripheral blood multinuclear cells were differentiated into macrophages. Gene expression also confirmed this trend, as RT-qPCR results after 7 days prednisolone showed significantly lower FcγR1 and FcγR3A mRNA expression. These data indicate a treatment that diminishes the surface expression of FcγRs and thus “primes” FcγRs for maximal aPD-1 efficacy.

### Example 9. Macrophage Removal of Anti-CD40 Monoclonal Antibodies

Dorsal skin fold chambers were installed on IL12-YFP reporter mice with MC38-H2B-mApple tumor cells injected. This system was used for longitudinal intravital imaging of dendritic cells (DCs) alongside tumor associated macrophages visualized by injection of Pacific Blue dye-labeled ferumoxytol nanoparticles. CD40 is a receptor found most abundantly on DCs and monoclonal antibodies against CD40 (aCD40) have been studied as promising anticancer immunotherapies. We labeled anti-mouse CD40 (clone FGK4.5) with AlexaFluor647 (aCD40), and monitored the distribution of this drug after i.v. delivery. Immediately following extravasation, the aCD40 was observed on DCs, however after 24 h, macrophage accumulation of aCD40 was observed despite the lower levels of CD40 on these cells. These data indicate that macrophage removal of monoclonal antibody drugs is relevant for other classes of monoclonal antibody drugs, including those targeted to antigen presenting cells.

### REFERENCES

1. D. R. Leach, M. F. Krummel, J. P. Allison, Enhancement of Antitumor Immunity by CTLA-4 Blockade, Science 271, 1734-1736 (1996).
2. D. M. Pardoll, The blockade of immune checkpoints in cancer immunotherapy, Nat. Rev. Cancer 12, 252-264 (2012).
3. S. L. Topalian, C. G. Drake, D. M. Pardoll, Immune checkpoint blockade: a common denominator approach to cancer therapy, Cancer Cell 27, 450-461 (2015).
4. K. M. Mahoney, P. D. Rennert, G. J. Freeman, Combination cancer immunotherapy and new immunomodulatory targets, Nat Rev Drug Discov 14, 561-584 (2015).
5. G. P. Dunn, L. J. Old, R. D. Schreiber, The Three Es of Cancer Immunoediting—Annual Review of Immunology, 22(1):329-360, Annu. Rev. Immunol. (2004).
6. R. D. Schreiber, L. J. Old, M. J. Smyth, Cancer Immunoediting: Integrating Immunity's Roles in Cancer Suppression and Promotion, Science 331, 1565-1570 (2011).
7. D. S. Chen, I. Mellman, Oncology meets immunology: the cancer-immunity cycle, Immunity 39, 1-10 (2013).
8. S. L. Topalian, F. S. Hodi, J. R. Brahmer, S. N. Gettinger, D. C. Smith, D. F. McDermott, J. D. Powderly, R. D. Carvajal, J. A. Sosman, M. B. Atkins, P. D. Leming, D. R. Spigel, S. J. Antonia, L. Horn, C. G. Drake, D. M. Pardoll, L. Chen, W. H. Sharfman, R. A. Anders, J. M. Taube, T. L. McMiller, H. Xu, A. J. Korman, M. Jure-Kunkel, S. Agrawal, D. McDonald, G. D. Kollia, A. Gupta, J. M. Wigginton, M. Sznol, Safety, activity, and immune correlates of anti-PD-1 antibody in cancer, N Engl J Med 366, 2443-2454 (2012).
9. E. B. Garon, N. A. Rizvi, R. Hui, N. Leighl, A. S. Balmanoukian, J. P. Eder, A. Patnaik, C. Aggarwal, M. Gubens, L. Horn, E. Carcereny, M.-J. Ahn, E. Felip, J.-S. Lee, M. D. Hellmann, O. Hamid, J. W. Goldman, J.-C. Soria, M. Dolled-Filhart, R. Z. Rutledge, J. Zhang, J. K. Lunceford, R. Rangwala, G. M. Lubiniecki, C. Roach, K. Emancipator, L. Gandhi, KEYNOTE-001 Investigators, Pembrolizumab for the treatment of non-small-cell lung cancer, N Engl J Med 372, 2018-2028 (2015).
10. P. C. Tumeh, C. L. Harview, J. H. Yearley, I. P. Shintaku, E. J. M. Taylor, L. Robert, B. Chmielowski, M. Spasic, G. Henry, V. Ciobanu, A. N. West, M. Carmona, C. Kivork, E. Seja, G. Cherry, A. J. Gutierrez, T. R. Grogan, C. Mateus, G. Tomasic, J. A. Glaspy, R. O. Emerson, H. Robins, R. H. Pierce, D. A. Elashoff, C. Robert, A. Ribas, PD-1 blockade induces responses by inhibiting adaptive immune resistance, Nature 515, 568-571 (2014).
11. N. A. Rizvi, M. D. Hellmann, A. Snyder, P. Kvistborg, V. Makarov, J. J. Havel, W. Lee, J. Yuan, P. Wong, T. S. Ho, M. L. Miller, N. Rekhtman, A. L. Moreira, F. Ibrahim, C. Bruggeman, B. Gasmi, R. Zappasodi, Y. Maeda, C. Sander, E. B. Garon, T. Merghoub, J. D. Wolchok, T. N. Schumacher, T. A. Chan, Mutational landscape determines sensitivity to PD-1 blockade in non-small cell lung cancer, Science 348, 124-128 (2015).
12. T. N. Schumacher, R. D. Schreiber, Neoantigens in cancer immunotherapy, Science 348, 69-74 (2015).
13. M. S. Rooney, S. A. Shukla, C. J. Wu, G. Getz, N. Hacohen, Molecular and genetic properties of tumors associated with local immune cytolytic activity, Cell 160, 48-61 (2015).
14. C. Pfirschke, C. Engblom, S. Rickelt, V. Cortez-Retamozo, C. Garris, F. Pucci, T. Yamazaki, V. Poirier-Colame, A. Newton, Y. Redouane, Y.-J. Lin, G. Wojtkiewicz, Y. Iwamoto, M. Mino-Kenudson, T. G. Huynh, R. O. Hynes, G. J. Freeman, G. Kroemer, L. Zitvogel, R. Weissleder, M. J. Pittet, Immunogenic Chemotherapy Sensitizes Tumors to Checkpoint Blockade Therapy, Immunity 44, 343-354 (2016).
15. A. J. Minn, E. J. Wherry, Combination Cancer Therapies with Immune Checkpoint Blockade: Convergence on Interferon Signaling, Cell 165, 272-275 (2016).
16. T. F. Gajewski, H. Schreiber, Y.-X. Fu, Innate and adaptive immune cells in the tumor microenvironment, Nat. Immunol. 14, 1014-1022 (2013).
17. R. Noy, J. W. Pollard, Tumor-associated macrophages: from mechanisms to therapy, Immunity 41, 49-61 (2014).
18. C. Engblom, C. Pfirschke, M. J. Pittet, The role of myeloid cells in cancer therapies, Nat. Rev. Cancer 16, 447-462 (2016).
19. K. J. Oestreich, H. Yoon, R. Ahmed, J. M. Boss, NFATc1 Regulates PD-1 Expression upon T Cell Activation, The Journal of Immunology 181, 4832-4839 (2008).
20. S. Gilfillan, C. J. Chan, M. Cella, N. M. Haynes, A. S. Rapaport, K. S. Boles, D. M. Andrews, M. J. Smyth, M. Colonna, DNAM-1 promotes activation of cytotoxic lymphocytes by nonprofessional antigen-presenting cells and tumors, J. Exp. Med 205, 2965-2973 (2008).
21. T. R. Mempel, M. J. Pittet, K. Khazaie, W. Weninger, R. Weissleder, H. von Boehmer, U. H. von Andrian, Regulatory T Cells Reversibly Suppress Cytotoxic T Cell Function Independent of Effector Differentiation, Immunity 25, 129-141 (2006).
22. R. L. Reinhardt, H. E. Liang, K. Bao, A. E. Price, M. Mohrs, B. L. Kelly, R. M. Locksley, A Novel Model for IFNg-Mediated Autoinflammatory Syndromes, The Journal of Immunology 194, 2358-2368 (2015).
23. R. Weissleder, M. Nahrendorf, M. J. Pittet, Imaging macrophages with nanoparticles, Nat Mater 13, 125-138 (2014).
24. F. Nimmerjahn, J. V. Ravetch, Divergent immunoglobulin g subclass activity through selective Fc receptor binding, Science 310, 1510-1512 (2005).
25. R. Dahan, E. Sega, J. Engelhardt, M. Selby, A. J. Korman, J. V. Ravetch, FcγRs Modulate the Anti-tumor Activity of Antibodies Targeting the PD-1/PD-L1 Axis, Cancer Cell 28, 285-295 (2015).
26. P. V. Beum, A. D. Kennedy, M. E. Williams, M. A. Lindorfer, R. P. Taylor, The shaving reaction: rituximab/CD20 complexes are removed from mantle cell lymphoma and chronic lymphocytic leukemia cells by THP-1 monocytes, J Immunol. 176, 2600-2609 (2006).
27. M. J. Feige, S. Nath, S. R. Catharino, D. Weinfurtner, Structure of the Murine Unglycosylated IgG1 Fc Fragment, J. Mol. Bio 391, 599-608 (2009).
28. R. P. Taylor, M. A. Lindorfer, Fcγ-receptor-mediated trogocytosis impacts mAb-based therapies: historical precedence and recent developments, Blood 125, 762-766 (2015).
29. P. Bruhns, B. Iannascoli, P. England, D. A. Mancardi, N. Fernandez, S. Jorieux, M. Daeron, Specificity and affinity of human Fc receptors and their polymorphic variants for human IgG subclasses, Blood 113, 3716-3725 (2009).
30. A. Lux, X. Yu, C. N. Scanlan, F. Nimmerjahn, Impact of Immune Complex Size and Glycosylation on IgG Binding to Human Fc Rs, The Journal of Immunology 190, 4315-4323 (2013).
31. C. Wang, K. B. Thudium, M. Han, X.-T. Wang, H. Huang, D. Feingersh, C. Garcia, Y. Wu, M. Kuhne, M. Srinivasan, S. Singh, S. Wong, N. Garner, H. Leblanc, R. T. Bunch, D. Blanset, M. J. Selby, A. J. Korman, In vitro characterization of the anti-PD-1 antibody nivolumab, BMS-936558, and in vivo toxicology in non-human primates, Cancer Immunol Res 2, 846-856 (2014).
32. G. Suntharalingam, M. R. Perry, S. Ward, S. J. Brett, A. Castello-Cortes, M. D. Brunner, N. Panoskaltsis, Cytokine storm in a phase 1 trial of the anti-CD28 monoclonal antibody TGN1412, N Engl J Med 355, 1018-1028 (2006).
33. K. Hussain, C. E. Hargreaves, A. Roghanian, R. J. Oldham, H. T. C. Chan, C. I. Mockridge, F. Chowdhury, B. Frendeus, K. S. Harper, J. C. Strefford, M. S. Cragg, M. J. Glennie, A. P. Williams, R. R. French, Upregulation of Fc RIth on monocytes is necessary to promote the superagonist activity of TGN1412, Blood 125, 102-110 (2015).
34. V. Goede, K. Fischer, R. Busch, A. Engelke, B. Eichhorst, C. M. Wendtner, T. Chagorova, J. de la Serna, M.-S. Dilhuydy, T. Illmer, S. Opat, C. J. Owen, O. Samoylova, K. A. Kreuzer, S. Stilgenbauer, H. Döhner, A. W. Langerak, M. Ritgen, M. Kneba, E. Asikanius, K. Humphrey, M. Wenger, M. Hallek, Obinutuzumab plus chlorambucil in patients with CLL and coexisting conditions, N Engl J Med 370, 1101-1110 (2014).
35. F. Sclafani, D. Gonzalez de Castro, D. Cunningham, S. Hulkki Wilson, C. Peckitt, J. Capdevila, B. Glimelius, S. Roselló Keränen, A. Wotherspoon, G. Brown, D. Tait, R. Begum, J. Thomas, J. Oates, I. Chau, FcγRIIa and FcγRllla polymorphisms and cetuximab benefit in the microscopic disease, Clin. Cancer Res. 20, 4511-4519 (2014).
36. O. Manches, G. Lui, L. Chaperot, R. Gressin, J.-P. Molens, M.-C. Jacob, J.-J. Sotto, D. Leroux, J.-C. Bensa, J. Plumas, In vitro mechanisms of action of rituximab on primary non-Hodgkin lymphomas, Blood 101, 949-954 (2003).
37. L. Arnould, M. Gelly, F. Penault-Llorca, L. Benoit, British Journal of Cancer—Abstract of article: Trastuzumab-based treatment of HER2-positive breast cancer: an antibody-dependent cellular cytotoxicity mechanism? British Journal of Cancer. 94, 259-267 (2006).
38. R. A. Clynes, T. L. Towers, L. G. Presta, J. V. Ravetch, Inhibitory Fc receptors modulate in vivo cytoxicity against tumor targets. Nat. Med. 6, 443-446 (2000).
39. A. Natarajan, A. T. Mayer, L. Xu, R. E. Reeves, J. Gano, S. S. Gambhir, Novel Radiotracer for ImmunoPET Imaging of PD-1 Checkpoint Expression on Tumor Infiltrating Lymphocytes, Bioconjug. Chem. 26, 2062-2069 (2015).
40. M. Hettich, F. Braun, M. D. Bartholoma, R. Schirmbeck, G. Niedermann, High-Resolution PET Imaging with Therapeutic Antibody-based PD-1/PD-L1 Checkpoint Tracers, Theranostics 6, 1629-1640 (2016).
41. T. Schlothauer, S. Herter, C. F. Koller, S. Grau-Richards, V. Steinhart, C. Spick, M. Kubbies, C. Klein, P. Umaña, E. Mössner, Novel human IgG1 and IgG4 Fc-engineered antibodies with completely abolished immune effector functions, Protein Eng. Des. Sel. 29, 457-466 (2016).
42. P. Sharma, S. Hu-Lieskovan, J. A. Wargo, A. Ribas, Primary, adaptive, and acquired resistance to cancer immunotherapy, Cancer Cell 168, 707-723 (2017).
43. W. Hugo, J. M. Zaretsky, L. Sun, C. Song, B. H. Moreno, S. Hu-Lieskovan, B. Berent-Maoz, J. Pang, B. Chmielowski, G. Cherry, E. Seja, S. Lomeli, X. Kong, M. C. Kelley, J. A. Sosman, D. B. Johnson, A. Ribas, R. S. Lo, Genomic and Transcriptomic Features of Response to Anti-PD-1 Therapy in Metastatic Melanoma, Cell 165, 35-44 (2016).
44. B. Lehmann, M. Biburger, C. Bruckner, A. Ipsen-Escobedo, S. Gordan, C. Lehmann, D. Voehringer, T. Winkler, N. Schaft, D. Dudziak, H. Sirbu, G.F. Weber, F. Nimmerjahn, Tumor location determines tissue-specific recruitment of tumor-associated macrophages and antibody-dependent immunotherapy response Science Immunology, Science Immunology 2, 1-11 (2017).
45. G. M. Thurber, K. S. Yang, T. Reiner, R. H. Kohler, P. Sorger, T. Mitchison, R. Weissleder, Single-cell and subcellular pharmacokinetic imaging allows insight into drug action in vivo, Nat Commun 4, 1504 (2013).
46. G. R. Guile, P. M. Rudd, D. R. Wing, S. B. Prime, R. A. Dwek, A rapid high-resolution high-performance liquid chromatographic method for separating glycan mixtures and analyzing oligosaccharide profiles, Anal. Biochem. 240, 210-226 (1996).
47. R. J. Kurlander, J. Hall, Comparison of intravenous gamma globulin and a monoclonal anti-Fc receptor antibody as inhibitors of immune clearance in vivo in mice, J. Clin. Invest. 77, 2010-2018 (1986).
48. K. S. Yang, R. H. Kohler, M. Landon, R. Giedt, R. Weissleder, Single cell resolution in vivo imaging of DNA damage following PARP inhibition, Sci. Rep. 5, 10129 DOI: 10.1038/srep10129 (2015).
49. P. V. Beum, E. M. Peek, M. A. Lindorfer, F. J. Beurskens, P. J. Engelberts, P. W. H. I. Parren, J. G. J. van de Winkel, R. P. Taylor, Loss of CD20 and bound CD20 antibody from opsonized B cells occurs more rapidly because of trogocytosis mediated by Fc receptor-expressing effector cells than direct internalization by the B cells, The Journal of Immunology 187, 3438-3447 (2011).
50. M. Alieva, L. Ritsma, R. J. Giedt, R. Weissleder, Imaging windows for long-term intravital imaging: General overview and technical insights: IntraVital: Vol 3, No 2, DOI: 10.4161/intv.29917 (2014).
51. T. R. Mempel, S. E. Henrickson, U. H. von Andrian, T-cell priming by dendritic cells in lymph nodes occurs in three distinct phases, Nature 427, 154-159 (2004).
52. F. Nimmerjahn, P. Bruhns, K. Horiuchi, J. V. Ravetch, FcgammaRlV: a novel FcR with distinct IgG subclass specificity, Immunity 23, 41-51 (2005).

| Sequences | |
|---|---|
| IgG1 Mutation of N297 to Alanine | |
| (SEQ ID NO: 1) | |
| 221 | D K T H T C P P C P A P E L L G G P S V F L F P P K P K D T L M |
| 253 | I S R T P E V T C V V V D V S H E D P E V K F N W Y V D G V E V |
| 285 | H N A K T K P R E E Q Y A S T Y R V V S V L T V L H Q D W L N G |
| 317 | K E Y K C K V S N K A L P A P I E K T I S K A K G Q P R E P Q V |
| 349 | Y T L P P S R E E M T K N Q V S L T C L V K G F Y P S D I A V E |
| 381 | W E S N G Q P E N N Y K T T P P V L D S D G S F F L Y S K L T V |
| 413 | D K S R W Q Q G N V F S C S V M H E A L H N H Y T Q K S L S L S |
| 445 | P G K Stop |
| IgG1 Mutation of Leucine 234 to Alanine and Leucine 235 to Alanine | |
| (SEQ ID NO: 2) | |
| 221 | D K T H T C P P C P A P E A A G G P S V F L F P P K P K D T L M |
| 253 | I S R T P E V T C V V V D V S H E D P E V K F N W Y V D G V E V |
| 285 | H N A K T K P R E E Q Y N S T Y R V V S V L T V L H Q D W L N G |
| 317 | K E Y K C K V S N K A L P A P I E K T I S K A K G Q P R E P Q V |
| 349 | Y T L P P S R E E M T K N Q V S L T C L V K G F Y P S D I A V E |
| 381 | W E S N G Q P E N N Y K T T P P V L D S D G S F F L Y S K L T V |
| 413 | D K S R W Q Q G N V F S C S V M H E A L H N H Y T Q K S L S L S |
| 445 | P G K Stop |
| IgG1 Mutation of Proline 329 to Glycine | |
| (SEQ ID NO: 3) | |
| 221 | D K T H T C P P C P A P E L L G G P S V F L F P P K P K D T L M |
| 253 | I S R T P E V T C V V V D V S H E D P E V K F N W Y V D G V E V |
| 285 | H N A K T K P R E E Q Y N S T Y R V V S V L T V L H Q D W L N G |
| 317 | K E Y K C K V S N K A L G A P I E K T I S K A K G Q P R E P Q V |
| 349 | Y T L P P S R E E M T K N Q V S L T C L V K G F Y P S D I A V E |
| 381 | W E S N G Q P E N N Y K T T P P V L D S D G S F F L Y S K L T V |
| 413 | D K S R W Q Q G N V E S C S V M H E A L H N H Y T Q K S L S L S |
| 445 | P G K Stop |
| IgG1 Mutation of Leucine 235 to Glutamic Acid | |
| (SEQ ID NO: 4) | |
| 221 | D K T H T C P P C P A P E L E G G P S V F L F P P K P K D T L M |
| 253 | I S R T P E V T C V V V D V S H E D P E V K F N W Y V D G V E V |
| 285 | H N A K T K P R E E Q Y N S T Y R V V S V L T V L H Q D W L N G |
| 317 | K E Y K C K V S N K A L P A P I E K T I S K A K G Q P R E P Q V |
| 349 | Y T L P P S R E E M T K N Q V S L T C L V K G F Y P S D I A V E |
| 381 | W E S N G Q P E N N Y K T T P P V L D S D G S F F L Y S K L T V |
| 413 | D K S R W Q Q G N V E S C S V M H E A L H N H Y T Q K S L S L S |
| 445 | P G K Stop |
| IgG1 Mutation of Leucine 234 to Alanine, Leucine 235 to Alanine, and | |
| Proline 329 to Glycine | |
| (SEQ ID NO: 5) | |
| 221 | D K T H T C P P C P A P E A A G G P S V F L F P P K P K D T L M |
| 253 | I S R T P E V T C V V V D V S H E D P E V K F N W Y V D G V E V |
| 285 | H N A K T K P R E E Q Y N S T Y R V V S V L T V L H Q D W L N G |
| 317 | K E Y K C K V S N K A L G A P I E K T I S K A K G Q P R E P Q V |
| 349 | Y T L P P S R E E M T K N Q V S L T C L V K G F Y P S D I A V E |
| 381 | W E S N G Q P E N N Y K T T P P V L D S D G S F F L Y S K L T V |
| 413 | D K S R W Q Q G N V F S C S V M H E A L H N H Y T Q K S L S L S |
| 445 | P G K Stop |
| IgG2a Mutation of Valine 235 to Alanine, Glycine 237 to Alanine, | |
| Proline 238 to Serine, Histidine 268 to Alanine, Valine 309 to | |
| Leucine, Alanine 330 to Serine, and Proline 331 to Serine | |
| (SEQ ID NO: 6) | |
| 122 | A S T K G P S V F P L A P C S R S T S E S T A A L G C L V K D Y |
| 154 | F P E P V T V S W N S G A L T S G V H T F P A V L Q S S G L Y S |
| 186 | L S S V V T V P S S N F G T Q T Y T C N V D H K P S N T K V D K |
| 218 | T V E R K C C V E C P P C P A P P A A A S S V F L F P P K P K D |
| 250 | T L M I S R T P E V T C V V V D V S A E D P E V Q F N W Y V D G |
| 282 | V E V H N A K T K P R E E Q F N S T F R V V S V L T V L H Q D W |
| 314 | L N G K E Y K C K V S N K G L P S S I E K T I S K T K G Q P R E |
| 346 | P Q V Y T L P P S R E E M T K N Q V S L T C L V K G F Y P S D I |
| 378 | S V E W E S N G Q P E N N Y K T T P P M L D S D G S F F L Y S K |
| 410 | L T V D K S R W Q Q G N V F S C S V M H E A L H N H Y T Q K S L |
| 442 | S L S P G K |
| IgG4 Mutation of Serine 228 to Proline and Leucine 235 to Glutamic | |
| Acid | |
| (SEQ ID NO: 7) | |
| 222 | Y G P P C P P C P A P E F E G G P S V F L F P P K P K D T L M I |
| 254 | S R T P E V T C V V V D V S Q E D P E V Q F N W Y V D G V E V H |
| 286 | N A K T K P R E E Q F N S T Y R V V S V L T V L H Q D W L N G K |
| 318 | E Y K C K V S N K G L P S S I E K T I S K A K G Q P R E P Q V Y |
| 350 | T L P P S Q E E M T K N Q V S L T C L V K G F Y P S D I A V E W |
| 382 | E S N G Q P E N N Y K T T P P V L D S D G S F F L Y S R L T V D |
| 414 | K S R W Q E G N V F S C S V M H E A L H N H Y T Q K S L S L S P |
| 446 | G K Stop |
| IgG4 Mutation of Serine 228 to Proline, Leucine 235 to Glutamic Acid, | |
| and Proline 329 to Glycine | |
| (SEQ ID NO: 8) | |
| 222 | Y G P P C P P C P A P E F E G G P S V F L F P P K P K D T L M I |
| 254 | S R T P E V T C V V V D V S Q E D P E V Q F N W Y V D G V E V H |
| 286 | N A K T K P R E E Q F N S T Y R V V S V L T V L H Q D W L N G K |
| 318 | E Y K C K V S N K G L G S S I E K T I S K A K G Q P R E P Q V Y |
| 350 | T L P P S Q E E M T K N Q V S L T C L V K G F Y P S D I A V E W |
| 382 | E S N G Q P E N N Y K T T P P V L D S D G S F F L Y S R L T V D |
| 414 | K S R W Q E G N V F S C S V M H E A L H N H Y T Q K S L S L S P |
| 446 | G K Stop |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A modified anti-PD1, anti-PD-L1, or anti-CD40 IgG antibody with significantly reduced or abrogated Fc:FcγR binding interactions, wherein the antibody has one or more of:
(i) a modification to the primary sequence of Fc receptor to abrogate Fc receptor binding;
(ii) removal of N-linked Glycosylation on Fc Portion of Antibody to abrogate Fc receptor binding;
(iii) sialylation of Fc Portion of Antibody to abrogate Fc receptor binding; or
(iv) altered glycosylation of Fc Portion of Antibody to abrogate Fc receptor binding.

2. The modified antibody of claim 1, wherein the antibody is a IgG1 with one or more of a mutation of Asparagine 297 to Alanine (N297A); mutation of Leucine 234 to Alanine and Leucine 235 to Alanine (LALA mtutation); mutation of Proline 329 to Glycine (P329G); and/or mutation of Leucine 235 to Glutamic Acid (L235E).

3. The modified antibody of claim 2, wherein the antibody has a LALA mutation and mutation at P329G.

4. The modified antibody of claim 1, wherein the antibody is a IgG2 with one a mutation of Valine 234 to Alanine (V234A), Glycine 237 to Alanine (G237A), Proline 238 to Serine (P238S), Histidine 268 to Alanine (H268A), Valine 309 to Leucine, Alanine 330 to Serine (A330S), and Proline 331 to Serine (P331S) in the Fc Region (V234A/G237A/P238S/H268A/V309L/A330S/P331S).

5. The modified antibody of claim 1, wherein the antibody is a IgG1 with one or more of a mutation of Serine 228 to Proline and Leucine 235 to Glutamic Acid (L235E); mutation of Leucine 234 to Alanine and Leucine 235 to Alanine (LALA); and/or mutation of Serine 228 to Proline and Leucine 235 to Glutamic Acid (L235E) and Proline 329 to Glycine (P329G).

6. The modified antibody of claim 1, wherein N-linked Glycosylation on Fc Portion of Antibody was removed by digestion of N-linked glycan using Peptide:N-Glycosidase F (PNGase F).

7. The modified antibody of claim 1, which is an IgG1 that has been sialylated using chemoenzymatic glycosylation remodeling.

8. The modified antibody of claim 1, which is an IgG4 that exclusively contains GOF glycans at N297.

9. The modified antibody of claim 1, which comprises a modified anti-PD1 antibody, preferably selected from the group consisting of pembrolizumab, nivolumab, avelumab, pidilizumab, and atezolizumab; a modified anti-CD40 antibody, preferably selected from the group consisting of dacetuzumab, lucatumumab, bleselumab, teneliximab, ADC-1013, CP-870,893, Chi Lob 7/4, HCD122, SGN-4, SEA-CD40, BMS-986004, and APX005M; or an anti-PD-L1 antibody, preferably selected from the group consisting of BMS-936559, FAZ053, KN035, Atezolizumab, Avelumab, and Durvalumab.

10. A pharmacological composition comprising the modified antibody of claim 1, and a pharmaceutically acceptable carrier.

11. A method of treating a cancer in a subject, the method comprising administering a therapeutically effective amount of the modified antibody of claim 1 to a subject in need thereof.

12. A method of treating a cancer in a subject, the method comprising administering a therapeutically effective amount of the composition of claim 10 to a subject in need thereof.

13. A composition comprising an anti-PD1, anti-PD-L1, or anti-CD40 antibody and (i) an anti-Fc gamma receptor antibody or (ii) prednisolone.

14. A method of treating a cancer in a subject, the method comprising administering a therapeutically effective amount of an anti-PD1, anti-PD-L1, or anti-CD40 antibody and an anti-Fc gamma receptor antibody, preferably wherein the anti-Fc gamma receptor antibody engages activating Fc gamma receptors.

15. The method of claim 14, wherein the anti-Fc receptor antibody binds Fc gamma receptors I, II, and III.

16. The method of claim 14, wherein the anti-Fc receptor antibody is selected from the group consisting of antibodies that bind specifically to human Fc gamma receptor IIb, and antibodies that bind specifically to human Fc gamma receptor IIb.

17. (canceled)

18. A method of treating a cancer in a subject, the method comprising administering a therapeutically effective amount of an anti-PD1 anti-PD-L1, or anti-CD40 antibody and (i) prednisolone or (ii) an agent that reduces levels of tumor Fc gamma receptor expressing macrophages selected from small molecule or antibody inhibitors of CSF1 and small interfering RNA (siRNA) directed against CCR2.

19.-26. (canceled)
